(19) United States
(12) Patent Application Publication
(10) Pub. No.: US 2023/0044600 A1
(43) Pub. Date: Feb. 9, 2023

(54) In-vivo Continuous Directed Evolution System and Application Thereof

(71) Applicant: Jiangnan University, Wuxi (CN)

(72) Inventors: Jing Wu, Wuxi (CN); Zhanzhi Liu, Wuxi (CN); Xinyu Zhang, Wuxi (CN); Ying Xu, Wuxi (CN)

(21) Appl. No.: 17/935,605

(22) Filed: Sep. 27, 2022

Related U.S. Application Data
(63) Continuation of application No. PCT/CN2021/117642, filed on Sep. 10, 2021.

(30) Foreign Application Priority Data
Mar. 5, 2021 (CN) 2021102542635

(51) Int. Cl. C12N 15/10; C12N 15/64

(52) U.S. Cl. CPC C12N 15/102; C12N 15/64; C12N 15/70

(57) ABSTRACT

The disclosure discloses an in-vivo continuous directed evolution system and application thereof, and belongs to the fields of gene engineering and enzyme engineering. The system includes Escherichia coli host bacteria carrying a random mutation module mutagenesis plasmid, a programmed death module toxin-antitoxin system and a target gene expression module target plasmid. The modules are coupled with one another, and target genes are subjected to multiple rounds of continuous mutation by virtue of the random mutation module mutagenesis plasmid in the system, so that the mutation rate of the target genes is further increased, and ultimately, efficient evolution and screening of the target genes in the host bacteria are realized. According to the system, mutations are accurately positioned on the target genes, random mutations in non-target gene regions are reduced, and the system has good practical value and can be applied to directed evolution of various different functional proteins.

### REFERENCE TO SEQUENCE LISTING

The instant application contains a Sequence Listing in XML format as a file named “YGHY-2022-15-SEQ.xml”, created on Jul. 12, 2022, of 84 kB in size, and which is hereby incorporated by reference in its entirety.

### TECHNICAL FIELD

The disclosure relates to an in-vivo continuous directed evolution system and application thereof, and belongs to the fields of gene engineering and enzyme engineering.

### BACKGROUND

Directed evolution, also known as laboratory evolution, is a method of simulating the evolution process at the molecular level in a laboratory environment to ultimately obtain proteins with desired characteristics. However, the evolution process has the problems of low single-round mutation efficiency, heavy screening workload and complex screening methods, and several or even dozens of rounds of evolution are usually required to obtain ideal protein characteristics. Based on the above problems, the David Liu laboratory of the Harvard University puts forward a phage-assisted continuous evolution (PACE) system. According to the system, the directed evolution is coupled with the life cycle of M13 phages, and random mutations are generated on the target genes by combining mutagenic plasmids, so that the target genes are subjected to continuous evolution and directed screening. In PACE, the target genes replace genes pIII encoding the coat proteins of key phages and are integrated in the genomes of the M13 phages. The David Liu laboratory continuously provides fresh Escherichia coli host cells to the phages so as to prevent the accumulation of mutations of host genomes. The host cells contain the expression vectors of pIII, and the expression of pIII is coupled with some expected functions of related genes. The phages which cannot induce pIII to express are unable to reproduce and are flushed out of the system by continuous inflowing phages. The phages have the advantage of short generation time. A single cell can produce nearly 1000 progeny phages within 1 hour of infection with host bacteria. New phage particles appear after 10 minutes of infection. Each generation of phages represents a round of evolution. The system can complete dozens of rounds of evolution in one day, including replication, mutations and directed selection of the target genes. Compared with conventional methods, the system enables target proteins to generate new activity or improves the existing activity of the target proteins in a short time only with the minimum manual intervention. At present, the PACE system is successfully applied to directed evolution of T7 RNA polymerase (RNAp), and the T7 RNAp produces the recognition activity to a T3 promoter in a few days by using the PACE system.

Despite the above advantages, the PACE system is a phage based directed evolution system, and therefore, the PACE system has high requirements for equipment, operation of laboratory researchers and laboratory specification standards. In addition, the system has poor targeting on the target genes and is unable to accurately position mutations on the target genes, random mutations in non-target gene regions may have adverse effects on the physiological metabolism of hosts, and the universality of the PACE system is reduced. Therefore, the development of a novel efficient in-vivo continuous directed evolution system and the improvement of the targeting on the target genes have high theory and application significance.

### SUMMARY

### Technical Problem

An existing directed evolution system has poor targeting on target genes and is unable to accurately position mutations on the target genes. The existing directed evolution system has high requirements for laboratory specification standards and operation specifications and is not favorable for technical popularization and application.

### Technical Solution

A first objective of the disclosure is to provide an in-vivo continuous directed evolution system, wherein target proteins can be subjected to random mutations and directed screening in vivo by the system.

As a further improved solution of the disclosure, host cells of the system include, but are not limited to, Escherichia coli (E. coli).

As a further improved solution of the disclosure, host bacteria of the system include, two and more than two functional modules.

As a further improved solution of the disclosure, the functional modules include a random mutation module mutagenesis plasmid (MP), a programmed death module toxin-antitoxin system (TAS) and a target gene expression module target plasmid (TP), the random mutation module MP is coupled with the target gene expression module TP, and the target gene expression module TP is coupled with the programmed death module TAS.

In one embodiment, the coupling of the target gene expression module TP and the programmed death module TAS means that target genes on the TP module can induce the expression of antitoxic proteins on the TAS module after random mutations and is not limited to the following types: (1) DNA binding proteins: for example, the evolution of antitoxic protein transcription factors is coupled with the expression of the antitoxic proteins; (2) proteins acting on small molecules: for example, reaction products of part of carbohydrase, cutinase or other enzymes involved in metabolite or nonmetabolite biosynthesis are coupled with the expression of the antitoxic proteins; (3) fluorescent proteins: for example, the fluorescent proteins are coupled with the expression of the antitoxic proteins by using photosensitive promoters; and (4) nuclease such as Cas9: the functions of the Cas9 are coupled with the expression of the antitoxic proteins through corresponding gene control.

As a further improved solution of the disclosure, the random mutation module MP includes mutagenic genes and helper genes, the target gene expression module TP includes target genes and helper gene recognized or bound elements, and the programmed death module TAS includes toxin protein encoding genes and antitoxic protein encoding genes.

As a preferred solution of the disclosure, the mutagenic genes are selected from, but are not limited to, at least one of an encoding gene PolA of low-fidelity DNA polymerase I, an encoding gene AID of cytosine deaminase, an encoding gene APOBEC of cytosine deaminase and an encoding gene TadA of adenine deaminase.

The nucleotide sequence of the PolA is shown in SEQ ID NO: 7, the nucleotide sequence of the AID is shown in SEQ ID NO: 8, the nucleotide sequence of the APOBEC is shown in SEQ ID NO: 9, and the nucleotide sequence of the TadA is shown in SEQ ID NO: 10.

As a preferred solution of the disclosure, the helper genes are selected from, but are not limited to, one or more of an encoding gene of T7 RNAp, an encoding gene of nCas9 lacking the activity for cutting a non-complementary strand and an encoding gene of dCas9 only with the DNA binding capacity.

As a further improved solution of the disclosure, the target genes are encoding genes and/or non-encoding genes of one or more proteins.

As a preferred solution of the disclosure, the target genes include, but are not limited to, one or more of an encoding gene of T7 RNAp, a resistance gene of antibiotics, an encoding gene for decomposing enzymes in a metabolic pathway, an encoding gene for synthesizing the enzymes in the metabolic pathway, an encoding gene of DNA binding proteins, an encoding gene of nuclease, an encoding gene of carbohydrase and an encoding gene of protease.

As a preferred solution of the disclosure, the helper gene recognized or bound elements include, but are not limited to, a tac promoter, a pac promoter, an Sp6 promoter, an lac promoter, a T7 promoter, a pBAD promoter, a trc promoter, an npr promoter and sgRNA.

The nucleotide sequence of the tac promoter is shown in SEQ ID NO: 11, the nucleotide sequence of the pac promoter is shown in SEQ ID NO: 12, the nucleotide sequence of the Sp6 promoter is shown in SEQ ID NO: 13, the nucleotide sequence of the lac promoter is shown in SEQ ID NO: 14, the nucleotide sequence of the T7 promoter is shown in SEQ ID NO: 15, the nucleotide sequence of the pBAD promoter is shown in SEQ ID NO: 16, the nucleotide sequence of the trc promoter is shown in SEQ ID NO: 17, and the nucleotide sequence of the npr promoter is shown in SEQ ID NO: 18.

As a preferred solution of the disclosure, promoters for inducing the toxin protein encoding genes to express are inducible promoters and include, but are not limited to, a pBAD operating system, an Lac operating system, a Tac operating system and a Tet operating system, the antitoxic protein encoding genes are recognized and expressed after being subjected to directed evolution by the target genes, and the TAS further includes proteins assisting in recognition or binding according to requirements of different target proteins.

As a preferred solution of the disclosure, the toxin proteins encoding genes include, but are not limited to, YdfD, etc. capable of causing cell rupture, PezT, SezT, zeta toxin, etc. capable of repressing cytomembrane formation, FicT, CcdB, etc. capable of inhibiting DNA replication, and TacT, etc. capable of inhibiting translation, and the antitoxic proteins encoding genes are selected from DicB/SulA, PezA, SezA, epsilon antitoxin, FicA, CcdA, TacA etc. corresponding to the toxic proteins.

The amino acid sequence of the YdfD is shown in SEQ ID NO: 19, the amino acid sequence of the PezT is shown in SEQ ID NO: 20, the amino acid sequence of the SezT is shown in SEQ ID NO: 21, the amino acid sequence of the zeta toxin is shown in SEQ ID NO: 22, the amino acid sequence of the FicT is shown in SEQ ID NO: 23, the amino acid sequence of the CcdB is shown in SEQ ID NO: 24, the amino acid sequence of the TacT is shown in SEQ ID NO: 25, the amino acid sequence of the DicB is shown in SEQ ID NO: 26, the amino acid sequence of the SulA is shown in SEQ ID NO: 27, the amino acid sequence of the PezA is shown in SEQ ID NO: 28, the amino acid sequence of the SezA is shown in SEQ ID NO: 29, the amino acid sequence of the epsilon antitoxin is shown in SEQ ID NO: 30, the amino acid sequence of the FicA is shown in SEQ ID NO: 31, the amino acid sequence of the CcdA is shown in SEQ ID NO: 32, and the amino acid sequence of the TacA is shown in SEQ ID NO: 33.

In one embodiment, proteins assisting in recognition or binding include, but are not limited to, activated and inhibited transcription factors such as lacI, psiR, Lrp, LysG, PcaR, CadR, PadR, NanR, PcaU, BmoR, TtgR, EmrR, FdeR, FrmR, DmpR, BenR, FadR, SoxR, Alks and PobR.

The nucleotide sequence of the lacI is shown in SEQ ID NO: 34, the nucleotide sequence of the psiR is shown in SEQ ID NO: 35, the nucleotide sequence of the Lrp is shown in SEQ ID NO: 36, the nucleotide sequence of the LysG is shown in SEQ ID NO: 37, the nucleotide sequence of the PcaR is shown in SEQ ID NO: 38, the nucleotide sequence of the CadR is shown in SEQ ID NO: 39, the nucleotide sequence of the PadR is shown in SEQ ID NO: 40, the nucleotide sequence of the NanR is shown in SEQ ID NO: 41, the nucleotide sequence of the PcaU is shown in SEQ ID NO: 42, the nucleotide sequence of the BmoR is shown in SEQ ID NO: 43, the nucleotide sequence of the TtgR is shown in SEQ ID NO: 44, the nucleotide sequence of the EmrR is shown in SEQ ID NO: 45, the nucleotide sequence of the FdeR is shown in SEQ ID NO: 46, the nucleotide sequence of the FrmR is shown in SEQ ID NO: 47, the nucleotide sequence of the DmpR is shown in SEQ ID NO: 48, the nucleotide sequence of the BenR is shown in SEQ ID NO: 49, the nucleotide sequence of the FadR is shown in SEQ ID NO: 50, the nucleotide sequence of the SoxR is shown in SEQ ID NO: 51, the nucleotide sequence of the Alks is shown in SEQ ID NO: 52, and the nucleotide sequence of the PobR is shown in SEQ ID NO: 53.

In one embodiment, the expression vectors of the random mutation module MP, the target gene expression module TP and the programmed death module TAS include, but are not limited to, pET series, or pSB1C3, or pRSFDuet or pCDFDuet plasmids.

In one embodiment, the expression vectors of the random mutation module MP, the target gene expression module TP and the programmed death module TAS are different from one another.

A second objective of the disclosure is to provide a continuous directed evolution method of genes, and the continuous directed evolution system is transformed to microbial cells.

In one embodiment, the microbial cells include, but are not limited to, E. coli.

In one embodiment, according to the method, the microbial cells are induced by virtue of inducers, the random mutation module MP is induced to express mutagenic proteins, the target gene expression module TP is induced to express target proteins, the programmed death module TAS is induced to express proteins assisting in recognition or binding and toxin proteins, and corresponding substrates need to be added according to different target proteins.

In one embodiment, the induced microbial cells are transferred or continuously cultured in a culture medium containing inducers.

In one embodiment, the inducers in the method include, but are not limited to, inducers IPTG and L-Arabinose (L-ara).

The disclosure further protects the effect of the continuous directed evolution system or method described above in protein modification.

### Beneficial Effect

The in-vivo continuous directed evolution system of the disclosure has targeting on the target genes, mutagenic proteins can accurately recognize and mediate the target genes to generate random mutations, and the mutations are accurately positioned on the target genes. The system is suitable for screening in E. coli and is convenient to popularize and use in most laboratories, and selected action objects are wide.

### BRIEF DESCRIPTION OF FIGURES

FIG. 1 is a schematic flow diagram of in-vivo continuous directed evolution.

FIG. 2 is a plasmid profile of the random mutation module MP.

FIG. 3 is a plasmid profile of the target gene expression module TP.

FIG. 4 is a growth curve comparison diagram of the lethal effect of the programmed death module TAS.

FIG. 5 is a cell staining comparison diagram of the lethal effect of the programmed death module TAS.

FIG. 6 is a comparison diagram of colony forming units formed by evolved and screened products and a control group growing on a screening plate.

### DETAILED DESCRIPTION

The disclosure may be further described in details with reference to embodiments and drawings.

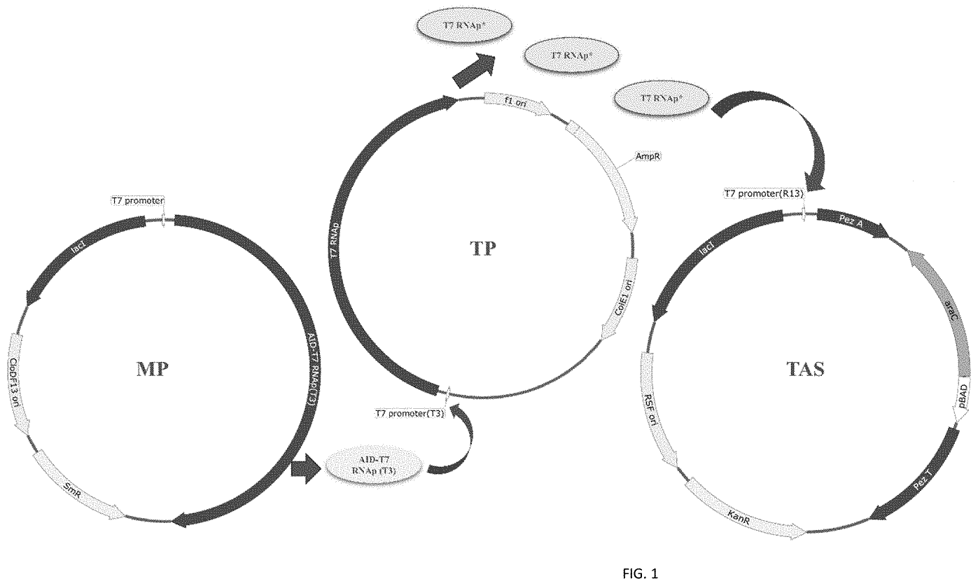

As shown in FIG. 1, a continuous directed evolution system in E. coli of one embodiment of the disclosure includes a random mutation module MP, a target gene expression module TP and a programmed death module TAS. The expression of antitoxic proteins in the programmed death module TAS is coupled with the functions of target proteins after mutations in the target gene expression module TP, i.e. the successfully evolved target proteins can directly start or control the expression of the antitoxic proteins in the TAS, or the successfully evolved target proteins can decompose or synthesize a substance to start or control the expression of the antitoxic proteins in the TAS. In the example in FIG. 1, an obtained T7 RNAp mutant can specifically recognize a T7 (R13) promoter of the antitoxic proteins and start the transcription of the antitoxic proteins to antagonize the effects of toxin proteins on host bacteria. The T7 RNAp mutant without mutations or advantages cannot recognize the T7 (R13) promoter. Thus, the antitoxic proteins cannot be normally expressed. The toxin proteins cannot be antagonized, and cell lysis is caused by the toxin proteins.

Those skilled in the art should understand that the disclosure is not limited to examples using T7 RNAp encoding genes as directed evolution, and any other similar technical solution is suitable for the disclosure. According to different target genes such as a carbohydrase gene, a cutinase gene, an esterase gene, a fluorescent protein gene, a nuclease gene and the like, the functions of the genes can be coupled with the expression of the antitoxic proteins in the programmed death module TAS by different principles.

The disclosure will list several examples of the target genes which can be applied to continuous directed evolution. It should be clear that a plurality of target genes can be selected in the disclosure and are not limited to the following types: (1) DNA binding proteins: for example, the evolution of antitoxic protein transcription factors is coupled with the expression of the antitoxic proteins; (2) proteins acting on small molecules: for example, reaction products of part of carbohydrase, cutinase or other enzymes involved in metabolite or non-metabolite biosynthesis are coupled with the expression of the antitoxic proteins; (3) fluorescent proteins: for example, the fluorescent proteins are coupled with the expression of the antitoxic proteins by using photosensitive promoters; and (4) nuclease such as Cas9: the functions of the Cas9 are coupled with the expression of the antitoxic proteins through corresponding gene control.

It should be noted that the programmed death module TAS in FIG. 1 is only one example. According to different mechanisms of toxicity of the toxin proteins in the TAS, a plurality of toxin protein-antitoxic protein systems suitable for the disclosure can be selected and are not limited to the following types: (1) toxin proteins capable of inhibiting DNA replication and transcription such as FicT/FicA from P. aeruginosa: the FicT modifies DNA gyrase and topoisomerase IV through adenylation, so that cell DNA is knotted, linked and loosened, and thus, reversible cells stop growing; (2) toxin proteins capable of inhibiting translation such as TacT/TacA from Salmonella: the TacT is acetyltransferase and can block primary amine groups of amino acid on charged tRNA molecules, so that translation is inhibited, and the formation of persister cells is promoted; (3) toxin proteins influencing cell division such as YdfD from E. coli: the YdfD can dissolve 99.9% of cells within 2 hours of induction, but SulA of a cell division inhibitor can eliminate cell lysis induced by the YdfD; and (4) toxin proteins influencing peptidoglycan synthesis such as SezT/SezA from Streptococcus suis Serotype and Pez A/T from Streptococcus pneumoniae: the peptidoglycan synthesis can be inhibited, and bacterial autolysis is ultimately caused. In addition, any toxin protein which can cause cell death and any antitoxic protein which can antagonize the toxin protein can be used as the programmed death module in the disclosure.

In one example of the disclosure, mutagenic proteins responsible for mutations are activation-induced cytidine deaminase (AID). The AID can act on single-stranded DNA, so that cytosine is deaminated and transformed into uracil and is ultimately transformed into thymine. T7 RNAp and AID are subjected to fusion expression and are responsible for enabling the AID to be bound with the downstream of the T7 promoter on the target gene expression module TP in a targeted manner, so that the mutagenic proteins have targeting on target genes. Except for the example, other mutagenic proteins with targeting, mutagenic protein mutants or mutagenic fusion proteins can also be used as the spontaneous random mutation module MP in the disclosure. For example (but not limited to), DNA polymerase Pol I (D355A/E357A or D424A/I709N/A759R) as a low-fidelity DNA polymerase mutant can specifically recognize plasmids with ColE1 Ori and enables the nucleotide sequence with a certain length after the transcription start site to mutate.

According to a directed evolution method provided by the disclosure, the random mutation module MP in the system expresses the mutagenic proteins under the action of inducers and can recognize and mediate the target genes in the target gene expression module TP to generate random mutations. After mutations of the target genes, the inducers for inducing the toxin proteins to express are added to the system, so that the system starts directed screening. Successfully evolved target protein mutants can mediate the expression of the antitoxic proteins on the programmed death module TAS, so that the toxin proteins cannot act on host cells, and ultimately, only the host cells containing successfully evolved target genes can survive.

The feasibility of the disclosure is described through the following examples. It should be noted that the following examples are only exemplary, and are only intended to illustrate the feasibility of the disclosure and not intended to limit the protection scope of the disclosure.

Sources of plasmids and strains involved in the following examples are as follows:

pET20b, pRSFDuet and pCDFDuet plasmids are purchased from the TaKaRa (Dalian) Bioengineering Co., Ltd, and E. coli BL21 (DE3) is preserved by our laboratory.

Reagents and culture media involved in the following examples are as follows:

PrimeSTAR HS DNA polymerase, DNA Marker and Dpn I restriction enzymes involved in the following examples are purchased from the TaKaRa (Dalian) Bioengineering Co., Ltd.

LB culture medium: The LB culture medium includes 0.5% (W/V) yeast extract powder, 1% (W/V) tryptone and 1% (W/V) sodium chloride, and 2% (W/V) agar powder is added to obtain the LB solid culture medium.

TB culture medium: The TB culture medium includes 1.2% (W/V) of tryptone, 2.4% (W/V) of yeast extract powder, 0.4% (V/V) of glycerol and a phosphate buffer solution (17 mM KH₂PO₄ and 72 mM K₂HPO₄).

### Example 1: Construction of Random Spontaneous Mutation Module MP

The specific steps were as follows:

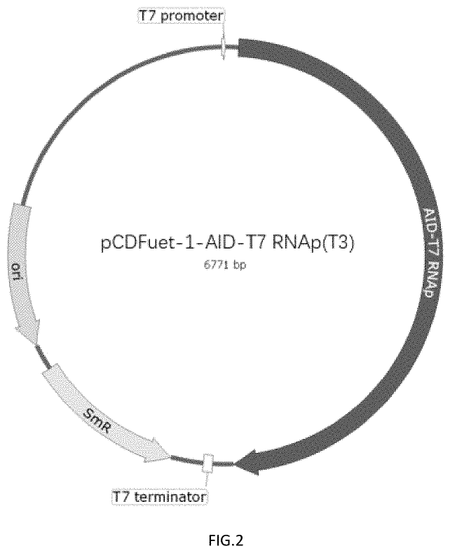

An AID-T7 RNAp mediated random spontaneous mutation module MP was taken for example. The fusion protein encoded AID-T7 RNAp (T3) (the amino acid sequence is shown in SEQ ID NO: 2) with the nucleotide sequence shown in SEQ ID NO: 1 was synthesized. T7 RNAp (T3) represented a T3 type mutant of T7 RNAp. In an enzyme cutting linkage manner, the AID-T7 RNAp (T3) was linked to an E. coli expression vector pCDFDuet to construct a recombinant vector pCDFDuet-pT7-AID-T7 RNAp (T3). A linkage product was transformed into E. coli JM109. The transformation product was spread on an LB solid culture medium (containing streptomycin with the final concentration of 50 μg·mL⁻¹). After inverted culture at 37° C. for 10-12 h, transformants on a plate were picked to be transferred to a LB liquid culture medium (containing streptomycin with the final concentration of 50 μg·mL⁻¹). After culture at 37° C. and 200 rpm for 12-14 h, plasmids were extracted for sequencing validation as one of the random mutation module MP and named pCDFuet-1-AID-T7 RNAp (T3) (as shown in FIG. 2).

### Example 2: Construction of Programmed Death Module TAS

The specific steps were as follows:

YdfD/SulA of a toxin protein-antitoxic protein system (TAS) was selected. The sequence thereof was synthesized. The amino acid sequences were shown in SEQ ID NO: 3 and SEQ ID NO: 4. The nucleotide sequences thereof were respectively introduced into two multiple cloning sites of pRSFDuet. The T7 promoter expressing toxin proteins in vectors was replaced with the pBAD operating system by using a MEGAWHOP cloning method. A PCR product was transformed into E. coli JM109 after being subjected to Dpn I digestion. The transformation product was spread on a LB solid culture medium (containing kanamycin with the final concentration of 50 μg·mL⁻¹). After inverted culture at 37° C. for 10-12 h, transformants on a plate were picked to be transferred to a LB liquid culture medium (containing kanamycin with the final concentration of 50 μg·mL⁻¹). After culture at 37° C. and 200 rpm for 12-14 h, plasmids were extracted and were sent to a sequencing company to be sequenced. The plasmids with correct sequencing results were recombinant vectors pRSFDuet-pT7-antitoxin-araC-pBAD-toxin.

### Example 3: Identification of Programmed Death Module TAS in E. coli

The specific steps were as follows:

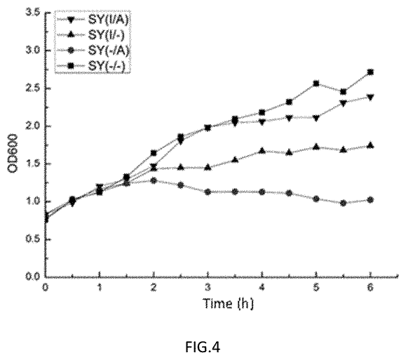

A toxin protein-antitoxic protein expression vector pRSFDuet-pT7-antitoxin-araC-pBAD-toxin was transformed into E. coli BL21 (DE3) and was transferred to a LB culture medium to induce fermentation. Firstly, glycerol bacteria preserved in a refrigerator of −80° C. were transferred to 10 mL of a LB culture medium (containing kanamycin with the final concentration of 50 μg·mL⁻¹) to be subjected to overnight culture at 37° C. and 200 rpm. A seed solution was transferred to 50 mL of a LB fermentation broth (containing kanamycin with the final concentration of 50 μg·mL⁻¹) in a proportion of 1:20, and inducers IPTG and L-ara were added to induce fermentation after 1.5 h at 37° C. and 200 rpm (in addition, a group only with IPTG, a group only with L-ara and a group without inducers were used as control groups). After the inducers were added, the growth situation of recombinant bacteria was determined. Results are shown in FIG. 4. When only the inducer L-ara was added, the expression of the toxin proteins was induced, and strain death was effectively mediated. When the inducers IPTG and L-ara were added at the same time, the inhibition of lac I to the T7 promoters was released by the IPTG, therefore, the T7 promoters were induced to start expression of the antitoxic proteins, and the strain growth was hardly influenced.

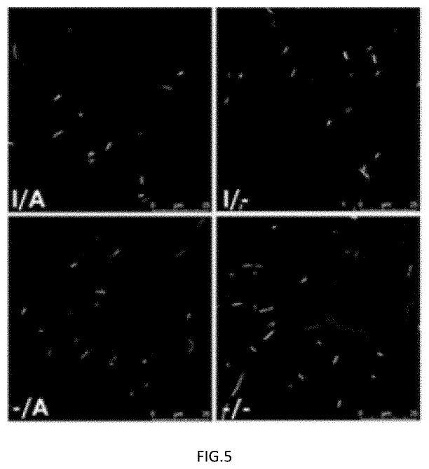

After induction for 6 h, bacterial cells were cleaned with normal saline and are resuspended. A PI/SYTO9 nucleic acid dye was added to a resuspension solution to dye. The life-or-death situation of the recombinant bacteria was observed and identified by using a confocal laser scanning microscope. Results are shown in FIG. 5. The dyeing experiment further proved that when only the inducer L-ara was added, the expression of the toxin proteins was induced by the pBAD operating system, and strain death was effectively mediated; when the inducers IPTG and L-ara were added at the same time, the inhibition of lac I to the T7 promoters was released by the IPTG, therefore, the T7 promoters were induced to start expression of the antitoxic proteins, and the strain growth was hardly influenced.

The experiments jointly proved that the expression of the toxic proteins and the antitoxic proteins in the programmed death module TAS in E. coli was controlled by the inducers IPTG and L-ara and the promoters.

By using the identification method of the example 3, the T7 promoters expressing the antitoxic proteins are mutated to T7 (R13) promoters by means of point mutation (the sequence of the T7 (R13) promoters refers to the article Meyer A J, Ellefson J W, Ellington A D. Directed Evolution of a Panel of Orthogonal T7 RNA Polymerase Variants for in Vivo or in Vitro Synthetic Circuitry [J]. ACS Synthetic Biology, 2015, 4(10).: Table 1 “P_{CGTA}”) so as to carry out subsequent implementation including target gene directed evolution.

### Example 4: Construction of Target Gene Expression Module TP in E. coli

The specific steps were as follows:

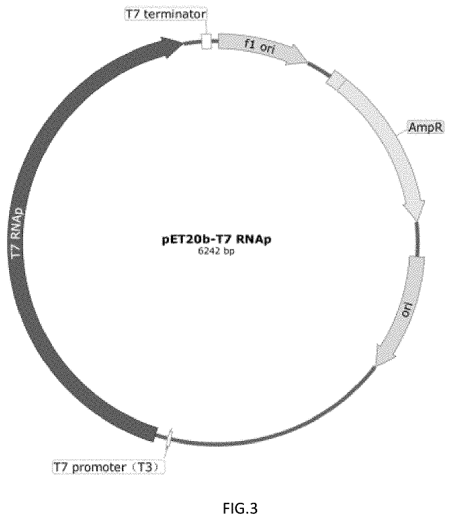

An E. coli BL21 (DE3) genome was used as a template, primers T7RNApRF-F and T7RNApRF-R were adopted for amplification to obtain T7 RNAp encoding genes as target genes, and the target genes were linked to E. coli expression vectors pET20b by using a MEGAWHOP cloning method. The recombinant expression vectors pET20b containing the T7 RNAp encoding genes were transformed into E. coli JM109 after being subjected to Dpn I digestion. The transformation product was spread on a LB solid culture medium (containing ampicillin with the final concentration of 100 μg·mL⁻¹). After inverted culture at 37° C. for 10-12 h, transformants on a plate were picked to be transferred to a LB liquid culture medium (containing ampicillin with the final concentration of 100 μg·mL⁻¹). After culture at 37° C. and 200 rpm for 12-14 h, plasmids were extracted for sequencing validation. The plasmids with correct sequencing results were recombinant vectors pET20b-T7 RNAp (as shown in FIG. 3). The T7 promoters on the recombinant vectors were mutated to T7 (T3) promoters by means of point mutation. The T7 (T3) promoters represented the promoters which could be recognized only by helper genes T7 RNAp (T3) on the random mutation module MP (the sequence of the T7 (T3) promoters refers to the article Meyer A J, Ellefson J W, Ellington A D. Directed Evolution of a Panel of Orthogonal T7 RNA Polymerase Variants for in Vivo or in Vitro Synthetic Circuitry [J]. ACS Synthetic Biology, 2015, 4(10).: Table 1 “P_{T3}”). The obtained recombinant plasmid pET20b-pT7 (T3)-T7 RNAp was the target gene expression module TP.

Primer T7RNApRF-F:
(SEQ ID NO 54)
CTTTAAGAAGGAGATATACATATGAACACGATTAACATCGC;
Primer T7RNApRF-R:
(SEQ ID NO: 55)
TGGTGGTGGTGGTGCTCGAGTTACGCGAACGCGAAGTCC.

### Example 5: Assembly and Characterization of All Modules in E. coli

The specific steps were as follows:

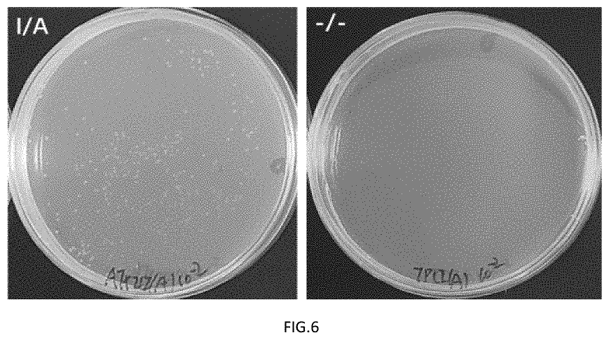

1) The random spontaneous mutation module MP, the target gene expression module TP and the programmed death module TAS were transferred to E. coli BL21 (DE3) in sequence to be subjected to overnight culture at 37° C. and 200 rpm. A seed solution was transferred to 50 mL of a TB fermentation broth in a proportion of 1:20. Inducers IPTG and L-ara (with the final concentrations of 0.4 mM and 0.3% respectively) were added to continuously induce fermentation after 1.5 h at 37° C. and 200 rpm. After an induced bacteria solution was diluted to a certain times, a plate was spread (containing 0.3% L-ara), and colony forming units were calculated (in addition, a group without L-ara was used as a control group). Results are shown in FIG. 6. Single colonies which could grow on the plate were subjected to target gene sequencing, and ultimately, an R13 type mutant T7 RNAp (R13) of T7 RNAp was obtained. Ultimately, the mutation efficiency was 4*10^{-5 to -6}/bp, the screening cycle was 6 days, and the physiological metabolism of mutant strains were not influenced.

2) The random spontaneous mutation module MP, the target gene expression module TP and the programmed death module TAS were transferred to E. coli BL21 (DE3) in sequence to be subjected to overnight culture at 37° C. and 200 rpm. A seed solution was transferred to 50 mL of a TB fermentation broth in a proportion of 1:20. Inducer IPTG (with the final concentration of 0.4 mM) was added to continuously induce fermentation after 1.5 h at 37° C. and 200 rpm. The inducer L-ara (with the final concentration of 0.3%) was added after fermentation for 2-4 h. After an induced bacteria solution was diluted to a certain times, a plate was spread (containing 0.3% L-ara), the colony forming units were calculated (in addition, a group without L-ara was used as a control group). Results are shown in FIG. 6. Single colonies which could grow on the plate were subjected to target gene sequencing, and ultimately, an R13 type mutant T7 RNAp (R13) of T7 RNAp was obtained. Ultimately, the mutation efficiency was 4*10^{-5 to -6}/bp, the screening cycle was 6 days, and the physiological metabolism of mutant strains were not influenced.

### Comparative Example 1

When the target gene expression module TP did not contain the target gene T7 RNAp, inducers were added to the recombinant bacteria to be subjected to continuous passage culture, then the bacteria solution was spread on a plate containing L-ara, and after overnight culture at 37° C., no colony grew.

### Comparative Example 2

When the random spontaneous mutation MP did not contain the mutagenic gene AID-T7 RNAp (T3), inducers were added to the recombinant bacteria to be subjected to continuous passage culture, then the bacteria solution was spread on a plate containing L-ara, and after overnight culture at 37° C., no colony grew.

### Comparative Example 3

When the target gene expression module TP did not contain a helper gene recognized or bound sequence, inducers were added to the recombinant bacteria to be subjected to continuous passage culture, then the bacteria solution was spread on a plate containing L-ara, and after overnight culture at 37° C., no colony grew.

The disclosure is illustrated through the examples that T7 RNAp recognizing the T7 promoters is evolved into a T7 RNAp mutant T7 RNAp (R13) recognizing the mutant type of the T7 (R13) promoters. T7 RNAp genes in TP are replaced with other target genes (such as carbohydrase genes, cutinase genes, nuclease genes, fluorescent protein genes and the like). The expression of antitoxin protein genes in TAS plasmids and the modification mode after expression are correspondingly adjusted, so that the expression of antitoxin proteins is coupled with the biological activity of new target genes on the TP, and the new target genes can be subjected to directed evolution by using the system. In addition, the replacement and modification of mutagenic protein encoding genes in the random mutation module MP is also included in the scope of the description of the disclosure.

### Example 6: Construction of In-Vivo Continuous Evolution System of D-psicose 3-epimerase (DPE)

Promoter-F-CTTACATTAATTGCGTTGCGCCCGCTTCTAGAGGAGCTGTTGAC (SEQ ID NO: 56) and promoter-R-GATATTTTTGCCGATCCCCATTGATCTTTTCTCCTCTTTTCCTCC (SEQ ID NO: 57) were used as upstream and downstream primers. pET20b-psir-ppsiA was used as a template. PCR was used for amplifying a transcription factor-promoter psir-ppsiA gene segment (the nucleotide sequence is shown in SEQ ID NO: 5). A PCR product was used as a Mega primer after being purified. The pRSFDuet-pT7-antitoxin-araC-pBAD-toxin in Example 2 was used as a template. Transcription factor-promoter psir-ppsiA was constructed to the pRSFDuet-pT7-antitoxin-araC-pBAD-toxin through MEGAWHOP to obtain a recombinant plasmid pRSFDuet-pT7-psir-ppsiA-antitoxin-araC-pBAD-toxin as the programmed death module TAS.

DPE-F-TAAGAAGGAGATATACATCGAGGATGAAACATGGCATCTATT (SEQ ID NO: 58) and DPE-R-CCTGGGCATGCCGCTTCAGTGGTGGTGGTGGTGGTG (SEQ ID NO: 59) were used as primers. pET20b-dpe was used as a template. The PCR amplification was used for obtaining encoding genes of DPE as target genes (the nucleotide sequence is shown in SEQ ID NO.NO: 6). The recombinant plasmid pET20b-pT7 (T3)-T7 RNAp obtained in Example 4 was used as a template. T7 RNAp was replaced with DPE by using a MEGAWHOP cloning method. A plasmid pET20b(+)-pT7(T3)-dpe containing the target genes of DPE was obtained as the target gene expression module TP.

pCDFuet-1-AID-T7 RNAp (T3) in Example 1 was used as a mutation plasmid MP. The MP, the TP and the TAS were co-transformed into host cells E. coli BL21 (DE3) to obtain recombinant bacteria as the in-vivo continuous evolution system of DPE.

### Example 7: In-Vivo Continuous Evolution Screening of DPEase

The recombinant bacteria obtained in Example 6 are inoculated into a shake flask containing 10 mL of a LB culture medium (containing 50 μg/mL Amp, 25 μg/mL Sm and 25 μg/mL Kana) and was cultured at 37° C. and 200 r/min for 8-10 h to obtain a seed solution. The seed solution was transferred to 20 mL of a TB culture medium (containing 50 μg/mL Amp, 25 μg/mL Sm and 25 μg/mL Kana) according to an inoculum size of 10% (v/v). After culture at 37° C. and 200 r/min for 1 h, an inducer isopropyl-β-D-thiogalactoside (IPTG) with the final concentration of 0.4 mmol/L was added. After culture at 25° C. and 200 r/min for 12 h, a culture solution was obtained. The culture solution was transferred to 20 mL of a new TB culture medium according to an inoculum size of 10% (v/v). The process lasts for a total of 8 rounds of induced fermentation. From the beginning of the 6^{th} round, L-ara with the final concentration of 3 mg/mL was added in the induction process to induce the expression of toxin proteins, and meanwhile, 0.5M D-fructose was added as a DPE substrate.

The culture solution with accumulation of mutations was inoculated to 50 mL of a 0.5 M D-fructose containing LB culture medium (containing 50 μg/mL Amp, 25 μg/mL Sm and 25 μg/mL Kana) according to an inoculum size of 5% (v/v). After culture at 37° C. and 200 r/min for 1 h, the inducers 0.4 mmol/L IPTG and 3 mg/mL L-ara were added. After culture at 25° C. and 200 r/min for 5.5 h, 1 ml of the uniformly mixed bacteria solution was taken and washed with 1 mL of sterile PBS and was centrifuged at 4° C. and 8000 r/min for 1 min, then supernate was removed, the bacteria solution was resuspended in 1 mL of PBS, and a bacterial suspension was diluted to a certain gradient with PBS. The bacteria solution diluted to a certain gradient was spread on an LB agar plate (containing 50 μg/mL Amp, 25 μg/mL Sm, 25 μg/mL Kana, 0.5 M D-fructose, 0.4 mmol/L IPTG and 3 mg/mL L-ara, and after culture at 37° C. for 12 h, a single clone of a mutant was obtained. After sequencing and shake flask validation, the mutant was determined as a mutant with obviously increased soluble expression.

Although the disclosure has been disclosed above with preferred examples, it is not intended to limit the disclosure. Those skilled in the art can make various changes and modifications without departing from the spirit and scope of the disclosure, and therefore, the protection scope of the disclosure should be defined by the claims.

What is claimed is:

1. A continuous directed evolution system, comprising a random mutation module mutagenesis plasmid, a programmed death module toxin-antitoxin system and a target gene expression module target plasmid, wherein the random mutation module mutagenesis plasmid comprises mutagenic genes and helper genes, the target gene expression module target plasmid comprises target genes and helper gene recognition or binding elements, and the programmed death module toxin-antitoxin system comprises toxin protein encoding genes and antitoxic protein encoding genes.

2. The system according to claim 1, wherein the mutagenic genes on the random mutation module mutagenesis plasmid comprise one or more of an encoding gene PolA of a low-fidelity DNA polymerase I mutant, an encoding gene AID of a cytosine deaminase, an encoding gene APOBEC of a cytosine deaminase and an encoding gene TadA of an adenine deaminase.

3. The system according to claim 1, wherein the helper genes comprise one or more of an encoding gene of a T7 RNA polymerase, an encoding gene of a nCas9 lacking the activity for cutting a non-complementary strand and an encoding gene of a dCas9 only with the DNA binding capacity.

4. The system according to claim 1, wherein the target genes are encoding genes and/or non-encoding genes of one or more proteins.

5. The system according to claim 1, wherein the target genes comprise one or more of an encoding gene of a T7 RNA polymerase, an antibiotic resistance gene, an encoding gene for decomposing enzymes in a metabolic pathway, an encoding gene for synthesizing the enzymes in the metabolic pathway, an encoding gene of DNA binding proteins, an encoding gene of a nuclease, an encoding gene of a carbohydrase and an encoding gene of a protease.

6. The system according to claim 1, wherein the helper gene recognition or binding elements comprise a tac promoter, a pac promoter, an Sp6 promoter, an lac promoter, a T7 promoter, a pBAD promoter, a trc promoter, an npr promoter and sgRNA.

7. The system according to claim 1, wherein promoters for inducing the toxin protein encoding genes to express are inducible promoters and comprise, but are not limited to, a pBAD operating system, an Lac operating system, a Tac operating system and a Tet operating system, the antitoxic protein encoding genes are recognized and expressed after being subjected to directed evolution by the target genes, and the toxin-antitoxin system further comprises proteins assisting in recognition or binding according to requirements of different target proteins.

8. The system according to claim 7, wherein the toxin protein encoding genes comprise YdfD capable of causing cell rupture, PezT, SezT and zeta toxin capable of repressing cytomembrane formation, FicT and CcdB capable of inhibiting DNA replication and TacT capable of inhibiting translation, and the antitoxic protein encoding genes are selected from DicB/SulA, PezA, SezA, epsilon antitoxin, FicA, CcdA and TacA genes corresponding to toxic proteins.

9. The system according to claim 7, wherein the proteins assisting in recognition or binding comprise one or more of activated and inhibited transcription factors lacI, psiR, Lrp, LysG, PcaR, CadR, PadR, NanR, PcaU, BmoR, TgtR, EmrR, FdeR, FrmR, DmpR, BenR, FadR, SoxR, Alks and PobR.

10. The system according to claim 1, wherein expression vectors of the random mutation module mutagenesis plasmid, the target gene expression module target plasmid and the programmed death module toxin-antitoxin system comprise pET series, or pSB1C3, or pRSFDuet or pCDFDuet plasmids.

11. A method of continuous directed evolution of genes, comprising transforming the continuous directed evolution system according to claim 1 to microbial cells, and the microbial cells comprises Escherichia coli.

12. The method according to claim 12, comprising inducing the microbial cells by virtue of inducers, inducing the random mutation module mutagenesis plasmid to express mutagenic proteins, inducing the target gene expression module target plasmid to express target proteins, inducing the programmed death module toxin-antitoxin system to express proteins assisting in recognition or binding and toxin proteins, and adding corresponding substrates according to different target proteins.

13. The method according to claim 12, comprising transforming the induced microbial cells and continuously culturing in a culture medium containing inducers.


SEQUENCE LISTING (59 sequences)

SEQ ID NO: 1 - DNA, 3273 bp, synthetic construct
  source 1..3273: mol_type = other DNA
atggatagcctgctgatgaatcgtcgcaaatttctgtatcagtttaaaaatgtgcgttgggccaaaggtcgtcgtgaaacctatctgtgctatgttgtgaaacgtcgtgatagcgcaaccagctttagcctggattttggttatctgcgcaataaaaatggttgtcatgtggagctgctgtttctgcgttatattagcgattgggatctggatccgggtcgttgttatcgtgttacctggtttaccagctggtcaccgtgttatgactgtgcacgtcatgttgcagattttctgcgtggtaatccgaatctgagcctgcgtatttttaccgcacgtctgtatttttgcgaagatcgtaaagcagaaccggaaggtctgcgtcgtctgcatcgtgcaggtgttcagattgccattatgacctttaaagattatttttattgctggaatacctttgtggaaaatcatgaacgcacctttaaagcatgggaaggcctgcatgaaaatagcgttcgtctgtctcgccagctgcgtcgtattctgctgccgctgtatgaagttgatgatctgcgtgatgcctttcgtaccctgggtttaaccggtcaggaagttgcatgcaccgccggcaacacgattaacatcgctaagaacgacttctctgacatcgaactggctgctatcccgttcaacactctggctgaccattacggtgagcgtttagctcgcgaacagttggcccttgagcatgagtcttacgagatgggtgaagcacgcttccgcaagatgtttgagcgtcaacttaaagctggtgaggttgcggataacgctgccgccaagcctctcatcactaccctactccctaagatgattgcacgcatcaacgactggtttgaggaagtgaaagctaagcgcggcaagcgcccgacagccttccagttcctgcaagaaatcaagccggaagccgtagcgtacatcaccattaagaccactctggcttgcctaaccagtgctgacaatacaaccgttcaggctgtagcaagcgcaatcggtcgggccattgaggacgaggctcgcttcggtcgtatccgtgaccttgaagctaagcacttcaagaaaaacgttgaggaacaactcaacaagcgcgtagggcacgtctacaagaaagcatttatgcaagttgtcgaggctgacatgctctctaagggtctactcggtggcgaggcgtggtcttcgtggcataaggaagactctattcatgtaggagtacgctgcatcgagatgctcattgagtcaaccggaatggttagcttacaccgccaaaatgctggcgtagtaggtcaagactctgagactatcgaactcgcacctgaatacgctgaggctatcgcaacccgtgcaggtgcgctggctggcatctctccgatgttccaaccttgcgtagttcctcctaagccgtggactggcattactggtggtggctattgggctaacggtcgtcgtcctctggcgctggtgcgtactcacagtaagaaagcactgatgcgctacgaagacgtttacatgcctgaggtgtacaaagcgattaacattgcgcaaaacaccgcatggaaaatcaacaagaaagtcctagcggtcgccaacgtaatcaccaagtggaagcattgtccggtcgaggacatccctgcgattgagcgtgaagaactcccgatgaaaccggaagacatcgacatgaatcctgaggctctcaccgcgtggaaacgtgctgccgctgctgtgtaccgcaaggacagggctcgcaagtctcgccgtatcagccttgagttcatgcttgagcaagccaataagtttgctaaccataaggccatctggttcccttacaacatggactggcgcggtcgtgtttacgccgtgtcaatgttcaacccgcaaggtaacgatatgaccaaaggactgcttacgctggcgaaaggtaaaccaatcggtaaggaaggttactactggctgaaaatccacggtgcaaactgtgcgggtgtcgataaggttccgttccctgagcgcatcaagttcattgaggaaaaccacgagaacatcatggcttgcgctaagtctccactggagaacacttggtgggctgagcaagattctccgttctgcttccttgcgttctgctttgagtacgctggggtacagcaccacggcctgagctataactgctcccttccgctggcgtttgacgggtcttgctctggcatccagcacttctccgcgatgctccgagatgaggtaggtggtcgcgcggttaacttgcttcctagtgagaccgttcaggacatctacgggattgttgctaagaaagtcaacgagattctacaagcagacgcaatcaatgggaccgataacgaagtagttaccgtgaccgatgagaacactggtgaaatctctgagaaagtcaagctgggcactaaggcactggctggtcaatggctggctcacggtgttactcgcagtgtgactaagcgttcagtcatgacgctggcttacgggtccaaagagttcggcttccgtcaacaagtgctggaagataccattcagccagctattgattccggcaagggtccgatgttcactcagccgaatcaggctgctggatacatggctaagctgatttgggaatctgtgagcgtgacggtggtagctgcggttgaagcaatgaactggcttaagtctgctgctaagctgctggctgctgaggtcaaagataagaagactggagagattcttcgcaagcgttgcgctgtgcattgggtaactcctgatggtttccctgtgtggcaggaatacaagaagcctattcagaagcgcttggacatgattttcctcggtcagttccgcttacagcctaccattaacaccaacaaagatagcgagattgatgcacgcaaacaggtgtctggtatcgctcctaactttgtacacagccaagacggtagccaccttcgtaagactgtagtgtgggcacacgagaagtacggaatcgaatcttttgcactgattcacgactccttcggtaccattccggctgacgctgcgaacctgttcaaagcagtgcgcgaaactatggttgacacatatgagtcttgtgatgtactggctgatttctacgaccagttcgctgaccagttgcacgagtctcaattggacaaaatgccagcacttccggctaaaggtaacttgaacctccgtgacatcttagagtcggacttcgcgttcgcgtaa

SEQ ID NO: 2 - protein, 1090 aa, synthetic construct
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKDYFYCWNTFVENHERTFKAWEGLHENSVRLSRQLRRILLPLYEVDDLRDAFRTLGLTGQEVACTAGNTINIAKNDFSDIELAAIPFNTLADHYGERLAREQLALEHESYEMGEARFRKMFERQLKAGEVADNAAAKPLITTLLPKMIARINDWFEEVKAKRGKRPTAFQFLQEIKPEAVAYITIKTTLACLTSADNTTVQAVASAIGRAIEDEARFGRIRDLEAKHFKKNVEEQLNKRVGHVYKKAFMQVVEADMLSKGLLGGEAWSSWHKEDSIHVGVRCIEMLIESTGMVSLHRQNAGVVGQDSETIELAPEYAEAIATRAGALAGISPMFQPCVVPPKPWTGITGGGYWANGRRPLALVRTHSKKALMRYEDVYMPEVYKAINIAQNTAWKINKKVLAVANVITKWKHCPVEDIPAIEREELPMKPEDIDMNPEALTAWKRAAAAVYRKDRARKSRRISLEFMLEQANKFANHKAIWFPYNMDWRGRVYAVSMFNPQGNDMTKGLLTLAKGKPIGKEGYYWLKIHGANCAGVDKVPFPERIKFIEENHENIMACAKSPLENTWWAEQDSPFCFLAFCFEYAGVQHHGLSYNCSLPLAFDGSCSGIQHFSAMLRDEVGGRAVNLLPSETVQDIYGIVAKKVNEILQADAINGTDNEVVTVTDENTGEISEKVKLGTKALAGQWLAHGVTRSVTKRSVMTLAYGSKEFGFRQQVLEDTIQPAIDSGKGPMFTQPNQAAGYMAKLIWESVSVTVVAAVEAMNWLKSAAKLLAAEVKDKKTGEILRKRCAVHWVTPDGFPVWQEYKKPIQKRLDMIFLGQFRLQPTINTNKDSEIDARKQVSGIAPNFVHSQDGSHLRKTVVWAHEKYGIESFALIHDSFGTIPADAANLFKAVRETMVDTYESCDVLADFYDQFADQLHESQLDKMPALPAKGNLNLRDILESDFAFA

SEQ ID NO: 3 - protein, 63 aa, synthetic construct
MNSAFVLVLTVFLVSGEPVDIAVSVHRTMQECMTAATEQKIPGNCYPVDKVIHQDNIEIPAGL

SEQ ID NO: 4 - protein, 170 aa, synthetic construct
MGYTSGYAHRSSSFSSAASKIARVSTENTTAGLISEVVYREDQPMMTQLLLLPLLQQLGQQSRWQLWLTPQQKLSREWVQASGLPLTKVMQISQLSPCHTVESMVRALRTGNYSVVIGWLADDLTEEEHAELVDAANEGNAMGFIMRPVSASSHATRQLSGLKIHSNLYH

SEQ ID NO: 5 - DNA, 1648 bp, synthetic construct
  source 1..1648: mol_type = other DNA
atgaccggtatctcttctaaaaaagctaccatctacgacctgtctatcctgtctggtgcttctgcttctaccgtttctgctgttctgaacggttcttggcgtaaacgtcgtatctctgaagaaaccgctgacaaaatcctgtctctggctaaagctcagcgttacaccaccaacttacaggctcgtggtctgcgttcttctaaatctggtctggttggtctgctggttccggtttacgacaaccgtttcttctcttctatggctcagaccttcgaaggtcaggctcgtaaacgtggtctgtctccgatggttgtttctggtcgtcgtgacccggaagaagaacgtcgtaccgttgaaaccctgatcgcttactctatcgacgctctgttcatcgctggtgttaccgacccggacggtgttcaccaggtttgcgctcgtgctgctctgccgcacgttaacatcgacctgccgggtaaattcgcttcttctgttatctctaacaaccgtcacggtgctgaaatcctgaccgctgctatcctggctcacgctgctaaaggtggttctctgggtccggacgacgttatcctgttcggtggtcacgacgaccacgcttctcgtgaacgtatcgacggtttccacgctgctaaagctgactacttcggtgttgaaggtggtgacgacatcgaaatcaccggttactctccgcacatgaccgaaatggctttcgaacgtttcttcggtcgtcgtggtcgtctgccgcgttgcttcttcgttaactcttctatcaacttcgaaggtctgctgcgtttcatgggtcgtcacgacggtgaagctttcggtgacatcgttgttggttgcttcgactacgacccgttcgcttctttcctgccgttcccggtttacatgatcaaaccggacatcgctcagatgctggaaaaaggtttcgaactgctggaagaaaaccgtaccgaaccggaagttaccatcatcgaaccgcagctgatcccgccgcgtaccgctctggaaggtccgctggacgacatctgggacccggttgctctgcgtcgtatggctaaataaagcaggcatgcccaggcatcaaataaaacgaaaggctcagtcgaaagactgggcctttcgttttatctgttgtttgtcggtgaacgctctctactagagtcacactggctcaccttcgggtgggcctttctgcgtttataacgtacgtacgtacgtggatcccttggagtataaatggtggctttttttgaacttatgcccgtcactgtgatctccccaactgattccgattattagagcacgcatccccttgacggaagggcgcttcatgatatggttattgcaccatcgattgtgcagattggcaatatcgattgtgcatggtggttgctatgggagtggcaagggagagtctcgaataagcgagatgagagattttgaacgcgtccgggaaaaacgggctgcgggcggatttcgtttgccgaatttttgaggaggaacatcaatgaagaaaattattgctgcggcggttggtctgtcgctggcgttgctctcatccgcagcctttgccgaagggccgaaggtgggcgtcgtcgtcaagatcggcggcattccgtggttcaacgcc

SEQ ID NO: 6 - DNA, 885 bp, synthetic construct
  source 1..885: mol_type = other DNA
atgaagcacggcatctactacgcctactgggagcaggagtgggaggccgactacaagtactacatcgagaaggtggccaagctgggcttcgacatcctggagatcgccgccagccccctgcccttctacagcgacatccagatcaacgagctgaaggcctgcgcccacggcaacggcatcaccctgaccgtgggccacggccccagcgccgagcagaacctgagcagccccgaccccgacatcaggaagaacgccaaggccttctacaccgacctgctgaagaggctgtacaagctggacgtgcacctgatcggcggcgccctgtacagctactggcccatcgactacaccaagaccatcgacaagaagggcgactgggagaggagcgtggagagcgtgagggaggtggccaaggtggccgaggcctgcggcgtggacttctgcctggaggtgctgaacaggttcgagaactacctgatcaacaccgcccaggagggcgtggacttcgtgaagcaggtggaccacaacaacgtgaaggtgatgctggacaccttccacatgaacatcgaggaggacagcatcggcggcgccatcaggaccgccggcagctacctgggccacctgcacaccggcgagtgcaacaggaaggtgcccggcaggggcaggatcccctgggtggagatcggcgaggccctggccgacatcggctacaacggcagcgtggtgatggagcccttcgtgaggatgggcggcaccgtgggcagcaacatcaaggtgtggagggacatcagcaacggcgccgacgagaagatgctggacagggaggcccaggccgccctggacttcagcaggtacgtgctggagtgccacaagcacagcctggag

SEQ ID NO: 7 - DNA, 2787 bp, synthetic construct
  source 1..2787: mol_type = other DNA
atggttcagatcccccaaaatccacttatccttgtagatggttcatcttatctttatcgcgcatatcacgcgtttcccccgctgactaacagcgcaggcgagccgaccggtgcgatgtatggtgtcctcaacatgctgcgcagtctgatcatgcaatataaaccgacgcatgcagcggtggtctttgacgccaagggaaaaacctttcgtgatgaactgtttgaacattacaaatcacatcgcccgccaatgccggacgatctgcgtgcacaaatcgaacccttgcacgcgatggttaaagcgatgggactgccgctgctggcggtttctggcgtagaagcggacgacgttatcggtactctggcgcgcgaagccgaaaaagccgggcgtccggtgctgatcagcactggcgataaagatatggcgcagctggtgacgccaaatattacgcttatcaataccatgacgaataccatcctcggaccggaagaggtggtgaataagtacggcgtgccgccagaactgatcatcgatttcctggcgctgatgggtgactcctctgataacattcctggcgtaccgggcgtcggtgaaaaaaccgcgcaggcattgctgcaaggtcttggcggactggatacgctgtatgccgagccagaaaaaattgctgggttgagcttccgtggcgcgaaaacaatggcagcgaagctcgagcaaaacaaagaagttgcttatctctcataccagctggcgacgattaaaaccgacgttgaactggagctgacctgtgaacaactggaagtgcagcaaccggcagcggaagagttgttggggctgttcaaaaagtatgagttcaaacgctggactgctgatgtcgaagcgggcaaatggttacaggccaaaggggcaaaaccagccgcgaagccacaggaaaccagtgttgcagacgaagcaccagaagtgacggcaacggtgatttcttatgacaactacgtcaccatccttgatgaagaaacactgaaagcgtggattgcgaagctggaaaaagcgccggtatttgcatttgataccgaaaccgacagccttgataacatctctgctaacctggtcgggctttcttttgctatcgagccaggcgtagcggcatatattccggttgctcatgattatcttgatgcgcccgatcaaatctctcgcgagcgtgcactcgagttgctaaaaccgctgctggaagatgaaaaggcgctgaaggtcgggcaaaacctgaaatacgctcgcggtattctggcgaactacggcattgaactgcgtgggattgcgtttgataccatgctggagtcctacattctcaatagcgttgccgggcgtcacgatatggacagcctcgcggaacgttggttgaagcacaaaaccatcacttttgaagagattgctggtaaaggcaaaaatcaactgacctttaaccagattgccctcgaagaagccggacgttacgccgccgaagatgcagatgtcaccttgcagttgcatctgaaaatgtggccggatctgcaaaaacacaaagggccgttgaacgtcttcgagaatatcgaaatgccgctggtgccggtgctttcacgcattgaacgtaacggtgtgaagatcgatccgaaagtgctgcacaatcattctgaagagctcacccttcgtctggctgagctggaaaagaaagcgcatgaaattgcaggtgaggaatttaacctttcttccaccaagcagttacaaaccattctctttgaaaaacagggcattaaaccgctgaagaaaacgccgggtggcgcgccgtcaacgtcggaagaggtactggaagaactggcgctggactatccgttgccaaaagtgattctggagtatcgtggtctggcgaagctgaaatcgacctacaccgacaagctgccgctgatgatcaacccgaaaaccgggcgtgtgcatacctcttatcaccaggcagtaactgcaacgggacgtttatcgtcaaccgatcctaacctgcaaaacattccggtgcgtaacgaagaaggtcgtcgtatccgccaggcgtttattgcgccagaggattatgtgattgtctcagcggactactcgcagaatgaactgcgcattatggcgcatctttcgcgtgacaaaggcttgctgaccgcattcgcggaaggaaaagatatccaccgggcaacggcggcagaagtgtttggtttgccactggaaaccgtcaccagcgagcaacgccgtagcgcgaaacggatcaactttggtctgatttatggcatgagtgctttcggtctggcgcggcaattgaacattccacgtaaagaagcgcagaagtacatggacctttacttcgaacgctaccctggcgtgctggagtatatggaacgcacccgtgctcaggcgaaagagcagggctacgttgaaacgctggacggacgccgtctgtatctgccggatatcaaatccagcaatggtgctcgtcgtgcagcggctgaacgtgcagccattaacgcgccaatgcagggaaccgccgccgacattatcaaacgggcgatgattgccgttgatgcgtggttacaggctgagcaaccgcgtgtacgtatgatcatgcaggtacacgatgaactggtatttgaagttcataaagatgatgttgatgccgtcgcgaagcagattcatcaactgatggaaaactgtacccgtctggatgtgccgttgctggtggaagtggggagtggcgaaaactgggatcaggcgcactaa

SEQ ID NO: 8 - DNA, 624 bp, synthetic construct
  source 1..624: mol_type = other DNA
atggatagcctgctgatgaatcgtcgcaaatttctgtatcagtttaaaaatgtgcgttgggccaaaggtcgtcgtgaaacctatctgtgctatgttgtgaaacgtcgtgatagcgcaaccagctttagcctggattttggttatctgcgcaataaaaatggttgtcatgtggagctgctgtttctgcgttatattagcgattgggatctggatccgggtcgttgttatcgtgttacctggtttaccagctggtcaccgtgttatgactgtgcacgtcatgttgcagattttctgcgtggtaatccgaatctgagcctgcgtatttttaccgcacgtctgtatttttgcgaagatcgtaaagcagaaccggaaggtctgcgtcgtctgcatcgtgcaggtgttcagattgccattatgacctttaaagattatttttattgctggaatacctttgtggaaaatcatgaacgcacctttaaagcatgggaaggcctgcatgaaaatagcgttcgtctgtctcgccagctgcgtcgtattctgctgccgctgtatgaagttgatgatctgcgtgatgcctttcgtaccctgggtttaaccggtcaggaagttgcatgcaccgccggc

SEQ ID NO: 9 - DNA, 687 bp, synthetic construct
  source 1..687: mol_type = other DNA
atgagtagcgaaaccggtccggttgcagtggatccgaccctgcgccgtcgcattgaaccgcacgagtttgaagtgttctttgatccgcgcgagctgcgcaaagaaacttgcctgctgtacgagattaactggggtggccgccatagcatctggcgccataccagccagaacaccaataagcacgtggaagtgaattttattgaaaaatttaccaccgagcgctacttctgccctaatacccgctgcagcatcacctggtttctgagctggagcccgtgcggcgaatgtagtcgcgccattaccgagttcctgagccgctatccgcatgtgaccctgttcatctacatcgcccgtctgtaccatcacgccgatccgcgcaatcgccaaggtctgcgtgatctgattagcagcggtgtgaccatccagatcatgaccgaacaagagagcggctattgctggcgcaacttcgtgaactattctccgagcaacgaagcccactggccgcgttatccgcatctgtgggtgcgcctgtatgtgctggagctgtactgcatcatcctgggcctgccgccttgcctgaatattctgcgccgtaaacagccgcaactgacattcttcaccatcgcactgcagagctgccattatcagcgcctgccgccgcacattttatgggccaccggtctgaaa

SEQ ID NO: 10 - DNA, 504 bp, synthetic construct
  source 1..504: mol_type = other DNA
ttgtctgaagtcgaatttagccacgaatactggatgcgtcacgcgctgacgctggcgaaacgtgcctgggatgagcgggaagtgccggtcggcgcggtattagtgcataacaatcgggtaatcggcgaaggctggaaccgcccgattggtcgccatgatcccaccgcacatgcagaaatcatggccctgcggcagggtggtctggtgatgcaaaattatcgtctgatcgacgccacgttgtatgtcacgcttgaaccatgtgtaatgtgtgccggagcgatgatccacagtcgcattggtcgcgtggtctttggtgcgcgtgacgcgaaaactggcgctgcgggatctttaatggatgtgctgcatcatccgggtatgaatcaccgagtggaaattacggaaggaatactggcggatgagtgcgcggcgttgctcagtgacttctttcgcatgcgccgccaggaaattaaagcgcagaaaaaagcgcaatcctcgacggattaa

SEQ ID NO: 11 - DNA, 29 bp, synthetic construct
  source 1..29: mol_type = other DNA
ttgacaattaatcatgggctcgtataatg

SEQ ID NO: 12 - DNA, 29 bp, synthetic construct
  source 1..29: mol_type = other DNA
tagataaaccgaagcttcgttgctagtat

SEQ ID NO: 13 - DNA, 19 bp, synthetic construct
  source 1..19: mol_type = other DNA
atttaggtgacactataga

SEQ ID NO: 14 - DNA, 78 bp, synthetic construct
  source 1..78: mol_type = other DNA
gacaccatcgaatggcgcaaaacctttcgcggtatggcatgatagcgcccggaagagagtcaattcagggtggtgaat

SEQ ID NO: 15 - DNA, 19 bp, synthetic construct
  source 1..19: mol_type = other DNA
taatacgactcactatagg

SEQ ID NO: 16 - DNA, 285 bp, synthetic construct
  source 1..285: mol_type = other DNA
aagaaaccaattgtccatattgcatcagacattgccgtcactgcgtcttttactggctcttctcgctaaccaaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccat

SEQ ID NO: 17 - DNA, 30 bp, synthetic construct
  source 1..30: mol_type = other DNA
ttgacaattaatcatccggctcgtataatg

SEQ ID NO: 18 - DNA, 569 bp, synthetic construct
  source 1..569: mol_type = other DNA
cagcagttcttttccgtcctctcttaagtaagcgctggtgaagtttgttgattgcacctggtgaataagttcaacagacactcccgccagcagcacaatccgcaatataacacccgccaagaacattgtgcgctgccggtttattttgggatgatgcaccaaaagatataagcccgccagaacaacaattgaccattgaatcagcagggtgctttgtctgcttaatataaaataacgttcgaaatgcaatacataatgactgaataactccaacacgaacaacaatcctttacttcttattaaggcctcattcggttagacagcggacttttcaaaaagtttcaagatgaaacaaaaatatctcatcttccccttgatatgtaaaaaacataactcttgaatgaaccaccacatgacacttgactcatcttgatattattcaacaaaaacaaacacaggacaatactatcaattttgtctagttatgttagtttttgttgagtattccagaatgctagtttaatataacaatataaagttttcagtattttcaaaaagggggatttatt

SEQ ID NO: 19 - protein, 63 aa, synthetic construct
MNTAFALVLTVFLVSGEPVDIAVSVHRTMQECVTAATEQKIPGNCYPVDKVIHQDNNEIPAGL

SEQ ID NO: 20 - protein, 252 aa, synthetic construct
MEIQDYTDSEFKHALARNLRSLTRGKKSSKQPIAILLGGQSGAGKTTIHRIKQKEFQGNIVIIDGDSFRSQHPHYLELQQEYGKDSVEYTKYFAGKMVESLVRELSHLGYNLLIEGTLRTIDVPKKTAQLLKSRGYEIQLALIATKPKLSYLSTLIRYEELYAINPNQARATPKEHHDFIVNHLVDNTRQLEELAIFERIQIYQRDRGCVYDAKENTTSAADVLQELLFGEWSQVEKDMLKSGEERLKDLTN

SEQ ID NO: 21 - protein, 258 aa, synthetic construct
MRLEEFSEAAFQKALQRTIRALIRGKTIPEQPKAILLGGQSGAGKTTIHRIKQKEFQGNIIIIDGDSYRSQHPNYLALQEKYGKDSVDYTKGFAGKMVEHLVDELSTQGYHLLIEGTLRTTQVPRQTAQLLASKGYQVSLAVIGTKPELSYLSTLIRYEELYAIDPNQARATPKEHHDGIVENLVDNLSELEREQLFDQIQIYQRDRTCIYDSEIDEGSAVEVLQECLFGQWSKVEEEMLKAGQERLGELVSANGEKK

SEQ ID NO: 22 - protein, 195 aa, synthetic construct
MKRLDLIVGPNGSGKTTFVAKFLAPLLHESVFVNADEIAKQRWPDEDPAPRAYEAARIAEQTRQALISQGRPFIAETVFSHPSKLDLIRSAQAADYTVRLLVLLVPEELTVQRVAARVEAGGHSVPIEKIRARYERLWPLVVDAIALADSSVVFDNSSEPGPRVVARMTRGQVVGTLTWPQWTPAPLWQRWTDSA

SEQ ID NO: 23 - protein, 200 aa, synthetic construct
MSDKFGEGRDPYLYPGLNVMRNRLGIHQAQRLAQAAYEMTALRAATIELGPLVRGLPHLCAIHRQLYQDIFDWAGQLREVDIYQGDTRFCHFAYIEKEGNALMQDLEEEGYLVGLAHEKFVERLAHYYCEINVLHPFRFGSGLAQRIFFEQLALHAGYALSWQGIAVETWKQANQSGAMGDLSALRAIFQKAISEARETE

SEQ ID NO: 24 - protein, 101 aa, synthetic construct
MQFKVYTYKRESRYRLFVDVQSDIIDTPGRRMVIPLASARLLSDKVSRELYPVVHIGDESWRMMTTDMASVPVSVIGEEVADLSHRENDIKNAINLMFWGI

SEQ ID NO: 25 - protein, 161 aa, synthetic construct
MGRVTAPEPLSAFHQVAEFVSGEAVLDDWLKQKGLKNQALGAARTFVVCKKDTKQVAGFYSLATGSVNHTEATGNLRRNMPDPIPVIILARLAVDLSFHGKGLGADLLHDAVLRCYRVAENIGVRAIMVHALTEEAKNFYIHHGFKSSQTQQRTLFLRLPQ

SEQ ID NO: 26 - protein, 63 aa, synthetic construct
MGKTLLPNVNTSEGCFEIGVTISNPVFTEDAINKRKQERELLNKICIVSMLARLRLMPKGCAQ

SEQ ID NO: 27 - protein, 170 aa, synthetic construct; identical to SEQ ID NO: 4

SEQ ID NO: 28 - protein, 159 aa, synthetic construct
MGIGKNIKSLRKTHDLTQPEFARIIGISRNSLSRYENGTSSVSTELIDIICQKFNVSYVDIVGEDKMLNPVEDYELTLKIEIVKERGANLLSRLYRYQDSQGISIDDESNPWILMSDDLSDLIHTNIYLVETFDEIERYSGYLDGIERMLEISEKRMVA

SEQ ID NO: 29 - protein, 169 aa, synthetic construct
MGKANMRRSLSMIGDNIKSLRRTHDLTQPEFAKMVGISRNSLSRYENGTSTVSTELIDRICQKFNVSYVDIVGEDKMLTPVEDYHLTLKVEVIKERGAAILSQLYRYQDSQDIAFDDESNPWILMSDDLAELINTKIYLVDTFDEIERYNGYLDGIERMLDMVHHRVVA

SEQ ID NO: 30 - protein, 119 aa, synthetic construct
MAAPVDRPTRVASDLLDSAAAEGARQSRSAKQQLDHWARLGREVSSQDNVSRRRVEAALSGQLSMRELTPEEGVVFNAEIEVELERRIAATHLQDELRAEGMRVVVLNDAGEIVQYPPA

SEQ ID NO: 31 - protein, 55 aa, synthetic construct
MKKLTDKQKSRFWEQRRNVNFQQSRRLEGIEIPLVTLTADEALVRLDELRRHYER

SEQ ID NO: 32 - protein, 73 aa, synthetic construct
MGKQRITVTVDSDSYQLLKAYDVNISGLVSTTLQNEARRLRAERWQAENQEGMAEVARFIEMNGSFADENRDW

SEQ ID NO: 33 - protein, 90 aa, synthetic construct
MGMKSDVQLNLRAKESQRALIDAAAEILHKSRTDFILETACQAAEKVILDRRVFNFNDEQYEEFINLLDAPVADDPVIEKLLARKPQWDV

SEQ ID NO: 34 - DNA, 1083 bp, synthetic construct
  source 1..1083: mol_type = other DNA
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggacatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 35 - DNA, 1080 bp, synthetic construct
  source 1..1080: mol_type = other DNA
atgaccggtatctcttctaaaaaagctaccatctacgacctgtctatcctgtctggtgcttctgcttctaccgtttctgctgttctgaacggttcttggcgtaaacgtcgtatctctgaagaaaccgctgacaaaatcctgtctctggctaaagctcagcgttacaccaccaacttacaggctcgtggtctgcgttcttctaaatctggtctggttggtctgctggttccggtttacgacaaccgtttcttctcttctatggctcagaccttcgaaggtcaggctcgtaaacgtggtctgtctccgatggttgtttctggtcgtcgtgacccggaagaagaacgtcgtaccgttgaaaccctgatcgcttactctatcgacgctctgttcatcgctggtgttaccgacccggacggtgttcaccaggtttgcgctcgtgctgctctgccgcacgttaacatcgacctgccgggtaaattcgcttcttctgttatctctaacaaccgtcacggtgctgaaatcctgaccgctgctatcctggctcacgctgctaaaggtggttctctgggtccggacgacgttatcctgttcggtggtcacgacgaccacgcttctcgtgaacgtatcgacggtttccacgctgctaaagctgactacttcggtgttgaaggtggtgacgacatcgaaatcaccggttactctccgcacatgaccgaaatggctttcgaacgtttcttcggtcgtcgtggtcgtctgccgcgttgcttcttcgttaactcttctatcaacttcgaaggtctgctgcgtttcatgggtcgtcacgacggtgaagctttcggtgacatcgttgttggttgcttcgactacgacccgttcgcttctttcctgccgttcccggtttacatgatcaaaccggacatcgctcagatgctggaaaaaggtttcgaactgctggaagaaaaccgtaccgaaccggaagttaccatcatcgaaccgcagctgatcccgccgcgtaccgctctggaaggtccgctggacgacatctgggacccggttgctctgcgtcgtatggctaaataa

SEQ ID NO: 36 - DNA, 495 bp, synthetic construct
  source 1..495: mol_type = other DNA
atggtagatagcaagaagcgccctggcaaagatctcgaccgtatcgatcgtaacattcttaatgagttgcaaaaggatgggcgtatttctaacgtcgagctttctaaacgtgtgggactttccccaacgccgtgccttgagcgtgtgcgtcggctggaaagacaagggtttattcagggctatacggcgctgcttaacccccattatctggatgcatcacttctggtattcgttgagattactctgaatcgtggcgcaccggatgtgtttgaacaattcaataccgctgtacaaaaacttgaagaaattcaggagtgtcatttagtatccggtgatttcgactacctgttgaaaacacgcgtgccggatatgtcagcctaccgtaagttgctgggggaaaccctgctgcgtctgcctggcgtcaatgacacacggacatacgttgttatggaagaagtcaagcagagtaatcgtctggttattaagacgcgctaa

SEQ ID NO: 37 - DNA, 921 bp, synthetic construct
  source 1..921: mol_type = other DNA
atgaacatcgagcatgtccgcgccctcgccgccgccgtcgacgaaggcaccgtcgaaggggccgcattcgtccttcggatcactccctcggcggcgagtcagcgcatccgggccctcgagtcccgcctcggccaggtcctcatccggcggacgaatcccatcaccgtcaccgaggcgggcaccgcggtcctccggtacgcccgccaggtcgagctcctcgaatccgaggccatggaccgcctccacgggatcgccgccgacgccgacccgcgggcgcccggcgtcctgcgcgagccgacagtgctgcggatcgggctcaacgccgattccctggcgacgtggttccgtcctgtcttcgccgaggtggccgcctgggacgatgtcgtcatccgcctcgatgtcgccgaccaggacaaggtcatgccgctgctcacctccggtgaggtgctcggcgcgatcgcctcgaccggggcgcgcacgtacggcacgcgcgtccagcgactcggcgcgatgcgctacgtcgccgtcgccgctcccgcactcctcgacaggcacggctggacgcagcgcgaggggtcgtcaggtcccttcgccgaggtggacctcgcgtccctgcccatggtcaacttcggcagggacgacgacctccagctcgccttcctcaaacgcaacggcatcaggcaggtgccgccgatgtcggtcgtgccgagctcgaccgaattcgcgaccgccgtcgtcgccggcgtcggctgggggatgatccccgtcctccagcttgccgacatcgacgccgaactcgtcccgatcactgcggatccgagcgtcgacgtccccctctactggcaccactggaagctcgcctcggacaagctctcccgcctcaccgaggcgctcacgagggccgccgcgcacatgcggtga

SEQ ID NO: 38 - DNA, 843 bp, synthetic construct
  source 1..843: mol_type = other DNA
atgaacgatcaattgcgcaactcctttgcatcagtggcgccgccgatcgtggcttcaccggccaagcgtatccaggcattcaccggtgatccggatttcatgacgtccctggcccgtggcctggccgtggtgcaggcgttccaggagcgcaagcgtcacctgaccatcgcccagatcagccaccgtaccgaaattccccgcgccgccgtgcggcgttgcctgcataccttgatcaagctcggctacgccaccacggacgggcgcacctattcgttgttgcccaaagtgctgaccctcggtcatgcctatctgtcgtccacaccgctggctgtctcggcccagccttatctggaccgcatgagcgagcaactgcatgaagcctgcaacatggcgaccctggagggcgatgacatcctttatatcgcccgttctgccactacccagcgcctgatctcggttgacctttcggtaggcgggcgcttgcctgcgtattgcacgtccatgggccgcattctgctggccgccctcgacgatgcctcgctgcaggattacctcgaccatgccgacctgcaaaccaagaccagccgcactctgaccacgccgcaagcgttatttgaatgcctgcaacaggtgcgtcagcagggctggtgcattgtcgaccaggaattggagcagggcctgcgttccatcgcggtgccggtgtatgacgcgtctggccaggtgttggccgcgctgaatgtcagcacccacgccgggcgggtcagccgcagcgagttggagcagcgctttttgccgagcatgctcagcgccagccgtgagctgagtgcgcaactgtttgcctaa

SEQ ID NO: 39 - DNA, 444 bp, synthetic construct
  source 1..444: mol_type = other DNA
atgaagatcggagaactggccaaggccaccgactgcgccgtggagacgatccgctattacgaacgtgagcacctgctgccggagccggcacgtaccgagggcaactatcggctctacacccaggcccacgtcgaacggctgaccttcatccgcaactgccgcacgctggacatgaccctggatgaaatccgcagcctgctacgtctgcgcgacagtcccgatgggtcgtgcggcagcgtcaatgcgttgatcgacgagcatatcgagcatgtgcaggcgcggatcgatggcttggtggcgctgcaggcgcagctggtggagctgcgtcagcgttgcaatgcgcaagggggagattgcgcgatcttgcagcagctcgaggtgaatggcgcggtatcggtgccggataccgagcattcgcatgtggggcgtagtcacgggcattga

SEQ ID NO: 40 - DNA, 549 bp, synthetic construct
  source 1..549: mol_type = other DNA
atgagagtattaaaatacgccatattagggcttttgcgaaaaggcgaattgagcggatatgatattacaagttattttaaagaggaactcggccaattttggagcgccaagcacagccagatttatcctgagctcaaaaagctgacggatgaagggtttattacattccggacaacaattcaaggcacaaagctggagaaaaagatgtacaccctgacagacagcggtaagcaggagctgcatgactggctgatccgccaccagccgataccggagacggtgaaggatgaatttatgctgaaggcatatttcatttcatctctatcgcggcaggaagcttctgatttgttcacagaccaactgctgaaacgcaaggccaagctgtctgatctgcagggaagctatgaaaagcttatggcttcagcggaaccgatgtcgttttcttcaccggacttcggccactatctcgtgctgacgaaagcgctggagcgggagaaaaattacgtttcttggctggaatcgattttagctatgatagatgaggattag

SEQ ID NO: 41 - DNA, 780 bp, synthetic construct
  source 1..780: mol_type = other DNA
atggtccctatgagcgcatttgatccacaagccgaagataccccgtcctccattggcaacagccttggccgccgtcctctggcgcgcaaaaaactgtctgaaatggtggaagaagagctggagcagatgatccggcgccgggagtttgcggagggtgaacagctgccgtccgaacgtgagctgatgaccttttttaacgttggacgtccttcggttcgtgaagcgctggcggcgctgaagcgtaagggactggtgcagatcagcaacggcgaacgcgctcgcgtttcccggccttcagcggacaccatcattggcgagctttccggcatggcgaaagactttttgtctcaccctggcggtatcgcgcattttgaacagctgcgtctgttttttgagtccagtctggttcgctatgccgcagaacacgccagcaatgagcagttggaaaagctgacgcaagcgctaaagatcaacagtcaatcgctggatgacaatgcgctgtttatccgctctgacgttgaatttcaccgcgtgcttgcggagatccccggtaatccaatctttatggctatccatgtggccctgctggactggctaatcgccgcccggccaacggtgcccgagcgcgaactgcacgaacataacagcgtcagctatcaacagcatatcgctattgttgatgctattcgtcgtcgcgatcctgacgaagccgatcgcgccctccagacccacctcaatagcgtatccgccacctggcacgccttaggtcagaaagcctga

SEQ ID NO: 42 - DNA, 813 bp, synthetic construct
  source 1..813: mol_type = other DNA
atgagtaaagatgaacggataatccagaatcaggacaacaaaaaaacgattcgtcatgaagattacattgctggtattggtaaaggcatggcgattttagatagctttagtagtaatcaacatcgtttaaacatttctatggcggctgaaaaaaccgggctcacccgagcagcagcaagacgccatttattaactttagaatatttaggatatcttgagtcagatggccactattactatttaactccaaaagtactcaaattttcaggagcttatttaggtgcagctcagcttccaaaagtatctcaacctttattaaatcttctaactaaccaaacctctctcatttattcggtgatggtacttgatggttatgaagccattactattgcccgtagtgctgctcaccaacaaactgaccgtgtaaatccatatggtttgcacttgggtaaccgcttacctgctcatgcaacctcggcgggtaaaattttactggcacatttaagccaagaagaacaattggactggctccaaaaatatccacttcagcgtttaactaaatatacgcatgtcgaaaatgataaatttttagagttactaaaagaaattagagaacaagattggtgttattcgcgtgaagaacacgaattaggtgttcatgcgctcgccgtaccgatttatggacaagactttaaagtggttgctgctttaaatattgtctctccaaccatgagaacaactgaagaatatttaattcaacatattctgccgctattgcaagagactgcgcgggatttaagacaagttctttaa

SEQ ID NO: 43 - DNA, 2010 bp, synthetic construct
  source 1..2010: mol_type = other DNA
atgtccaagatgcaagagttcgcgcggctggagacagtcgcgtcgatgcgcagagcggtctgggacggcaacgagtgtcagccggggaaagtggctgatgtcgttttgcgctcgtggacccggtgtcgtgctgaaggtgtcgttcccaatgcccgccaggagttcgacccgatcccgcgaacggcgcttgacgaaacggttgaggccaagcgggcgctgatccttgctgccgagccggtcgtcgacgcgttgatggagcagatgaacgacgcccccaggatgatcatcctgaacgacgaacggggcgtcgtgctgctgaaccagggaaacgacaccctccttgaagacgcccgccgccgggccgtgcgggtgggcgtctgctgggacgaacacgcccgaggcaccaatgccatgggaaccgcgctcgcggagaggaggcccgtagcgatccacggcgcagagcactacctcgagtcgaatacgattttcacctgcaccgcggcgccgatctacgatccgttcggcgagttcaccggaattctggatatcagcggatatgcgggggacatgggcccggttccgattccctttgttcagatggcggtgcaattcatcgagaatcagttgttccgccagacctttgccgattgcattctgctgcactttcatgtgcgccccgacttcgtcggaacgatgcgcgaagggatagccgtgctgtcgcgcgagggaaccatcgtctcgatgaaccgtgctgggctcaagatcgcagggctcaacctggaggccgtcgccgatcaccgtttcgattccgtcttcgacttgaatttcggggcctttctcgaccacgtgcggcagtccgccttcggtctcgtccgcgtctcgctctacggcggcgttcaggtctacgcccgagtggaaccgggcctgcgtgttccgccacgtccggccgcccacgcccgccctcctcggccggcaccgcggcctctggattcgctggacacgggcgacgcagcagtccgcctcgcgattgaccgcgcccgccgcgcgatcggccgcaacctcagcatcctcatccagggcgagacgggtgccggcaaggaagtgttcgccaagcatctgcatgccgagagcccgagaagcaaggggccgttcgttgccgtcaattgcgccgccatacccgagggtttgatcgagtccgagcttttcggatacgaagaaggggccttcactggcggacggcgcaaaggcaacatcggcaaggtcgcccaggcccacggcggcacgttgttcctcgacgagatcggagacatggcgccggggctgcagaccagactgctgcgggttctacaggaccgggcagtgatgcccctcggcggtcgcgaacctatgccggtcgacattgcgctggtatgcgcgacgcaccgcaatttgcgcagcctgatagcgcagggccagttccgcgaagacctctactaccgcctgaacgggctggcgatctcgctgccacccctgcgtcaacgtagcgaccttgccgccctggttaaccatatcctctttcagtgttgcgggggcgagccgcactacagcgtgagcccagaagtgatgacgctcttcaagcggcatgcgtggcccggcaacctacgccaactacataacgttctcgatgcagcgcttgccatgctcgacgacggccatgtcattgagccccatcacctccccgaagacttcgtcatggaggtcgattcgggcctccgaccgatcgaggaagacggttcgacggcggcgcatcgcgcgcgacagccggcgtcgggaagcggtcctgccaaaaagttgcaagatctcgcgttggatgccatcgagcaggcgatcgagcaaaacgagggaaatatatcggtcgccgcacggcagttgggggtcagccggaccacgatctaccgcaagctgaggcaactttcaccaaccggttgtcaccgaccggcacattggagccagtcgcggatcggcacatag

SEQ ID NO: 44 - DNA, 633 bp, synthetic construct
  source 1..633: mol_type = other DNA
atggtccgtcgaaccaaagaagaagcccaggaaacccgcgcccagatcatcgaggcggcggaaaaggccttctacaagcgcggggttgcgcgaaccaccctggccgacatcgccgagctggcgggtgtgacgcgcggggcgatctactggcacttcaacaacaaggcagagttggtgcaggcgctgctcgacagcctgcacgaaactcatgaccacttggcgcgggcgagtgaaagcgaggacgaagtcgacccgcttggctgcatgcgcaagctgctgctgcaagtgttcaacgagttggtgctcgatgcccgaacccggcgtatcaatgaaattctgcatcacaagtgcgagttcaccgatgacatgtgtgaaatccgccagcagcgccagggcgcggtactggattgccacaagggcattgccctggcgttggccaatgccgttcgccggggccagttgcctgacgcgctggatgtcgagcgcgcagcggtggcgatgtttgcctatgtcgatggcctgatcgggcgctggcttttgctgcctgacagtgtcgatctggcgggcgatgtcgaaaaatgggtcgacaccgggctggatatgctgcgcttgagcccggccctgcgcaaatga

SEQ ID NO: 45 - DNA, 531 bp, synthetic construct
  source 1..531: mol_type = other DNA
atggatagttcgtttacgcccattgaacaaatgctaaaatttcgcgccagccgccacgaagattttccttatcaggagatccttctgacccgtctttgcatgcatatgcagagcaagttgctggaaaaccgcaataaaatgctgaaggctcaggggattaacgagacgttgtttatggcgttgattacgctggagtctcaggaaaaccacagcattcagccttctgaattaagttgtgctcttggatcatcccgtaccaatgcgacgcgtattgccgatgaactggaaaaacgcggctggatcgaacgtcgtgaaagcgataacgatcgccgctgcctgcatctgcaattaacggaaaaaggtcacgagtttttgcgcgaggttttaccgccgcagcataactgcctgcatcaactctggtccgcgctcagcacaacagaaaaagatcagcttgagcaaatcacccgcaaattgctctcccgtctcgatcagatggaacaagacggtgcggttctcgaagcgatgagctaa

SEQ ID NO: 46 - DNA, 930 bp, synthetic construct
  source 1..930: mol_type = other DNA
atgcgtttcaacaagctcgacctcaatcttctggtcgccctggatgcactgctcacggagatgagcatcagccgcgccgccgaaaagatccatctgagccagtcggccatgagcaatgccctggcgcggctgcgcgagtatttcgatgatgaattgctgatccaggtgggccggcgcatggagcccacgccgcgcgccgaggtgctcaaggatgcggtgcatgatgtgctgcggcgtatcgatggctccatcgcggcgctgccggccttcgtgccggccgagtccacgcgcgagtttcgcatctcggtttcggactttacgctctccgtcctcatcccccgggtgctggcgcgcgcgcacgccgagggcaagcacatccgctttgccctgatgccgcaggtgcaagacccgacccgctcgctggatcgggccgaggtggacctgctggtcttgccgcaggaattctgcacgcccgatcatcctgccgaagaggtcttccgcgaacggcatgtctgcgtggtctggcgcgacagtgcgctggcgcaaggcgagctgacgctggaacgctacatggcctcaggccatgtggtgatggtgccgcctggggccaatgcgtcgtcggtggaggcgtggatggccaggaagctgggctttgcgcgccgggtggaagtgaccagcttcagcttcgcttctgcgctggcgctggtacaggggacggaccgcatcgccacggtgcatgcccggctggcgcagctgctggctccgcaatggccggtggtgatcaaggagagtccgctgtcgctgggcgagatgcggcagatgatgcagtggcatcgctaccgcagcaatgatcctggcatccagtggctgcgtcgggtgtttctggagagtgcgcaggagatggatgcggcgctgccaggcatctgctga

SEQ ID NO: 47 - DNA, 276 bp, synthetic construct
  source 1..276: mol_type = other DNA
atgcccagtactccggaagagaagaaaaaggtccttactcgagttcgtcgtattcgggggcagattgatgctctggaacggtcgctggagggtgatgccgaatgccgtgccatactccaacagatcgctgccgttcggggcgcggctaatgggctgatggcagaagtgcttgaaagccatatccgggaaacgtttgaccgaaatgactgctacagccgcgaagtcagccaatccgttgacgacactattgaactggttcgtgcctatcttaaatag

SEQ ID NO: 48 - DNA, 1704 bp, synthetic construct
  source 1..1704: mol_type = other DNA
atgaaagttgattacaaggcgtcggagcaccacccgaccctgaaggacctgaccgaccgggtccgcttcctcggcaacgagggcaagatctggctcgacgagcaacgcatgctcctgctgcaggcctccgccatgggctccttccgccgcgagctggtggagatgctcggcgtcgagcgggccaagggcctgttcctgcgcctgggctaccagtcggggctgaaggacgccgaactggcgcgcaagctgcgcccggacgccgacgcggtggagatgttcctcaccggcccgcagctgcattcgctcaagggcatggtcaaggtcgagccgatttcgatgaacatcgatatcgagaacggccagttctacgccgacctcgagtggcagaactcctacgaggtggacaactgccaggccgaaggcatgcagtccgaccagccggtgtgctggaccctgctcggctacgccatcagctattcgtcgttcttcctcggccgccaggtgctctacaaggaagtcagttgccgtggctgcggcgattcccgctgccggatcatcggcaagccggtggaggaatgggaggacgccggcgagttcctgcgctacttccagagcgacccgatcatcgacgaactgcaagccctgcaagtgcaggtggcgaccatgcgccagcgcctgcacgaggacgccggacagttctacggcatcggccaggcgccgctctaccgcagggcctgcaccctgatcgacaaggccgcgccgggcaaggcctcggtgctgctgctcggcgaaaccggcgtgggcaaggaagtcatggcgcgcagcctgcacctgcgcagcgagcgcgccggcgcgcccttcgtggcgctgaactgcgccgccattccaccggacctgatcgaggccgagctgttcggtgtcgagcgcggcgcctacaccggcgcccagcagtcgcgcatgggccgtttcgaacgggccaacggcggcaccctgttcctcgacgaagtggtggagctcacgccccgcgcccaggccagcctgctgcgcgtgctgcaggaggaagaactggagcgggtgggcgattcgcagacccgcaaggtggacgtgcgggtggtggccgccacccacgaggacctggcccgcgcggtgaaggacggccgcttccgcgccgacctctactaccggctgaacgtctacccggtgttcatcccctcgctgcgcgagcgcaaggaagacattccgctgctggtggagcacttcctctcgcgcctgcacgaccgctacggcaagaccaccctggggctctccgaccgcgccctggaagcctgcctgcgctacgactggccgggcaacatccgcgagctggagaacctgattgagcgcggcgtgatcatcaccgacgccaaccagagcatcagcgccgacgccctcttcccgcaccagtccgacgaagcccagggcatcggcctgtccagcgagggcgcactggtggagacctgcgccagcgtcgagccggactgggtcacccgcctgatcgacagcggcctgagcctggacgcgctggaggaagccctgatgcagggagcgatgaagcgggcgcagaacaacgtgtcggaagccgcgcgcctgctcggcatgacccgccccgccctggcctaccggctgaagaaactgccggccgaggatgccagcggccagcgctga

SEQ ID NO: 49 - DNA, 957 bp, synthetic construct
  source 1..957: mol_type = other DNA
atggaaagccgcctgctgagcgagcgaagtagcgtgtttcaccacgccgacccttatgctgtgtccgattatgtgaaccagcatgtaggccagcattgcatcggtctgtcccgtaccacccatccccaggctagcctcagccaccgcaagtttgccgagctcgacctgtgccgtatcagctatggcggcagtgttcgcgtgacttctccagcgctggaaaccatttatcacctgcaagtgttgctcaacggcaactgcctgtggcgtgggcacaagcacgagcagcacctggtgccgggcgagctgctgctgatcaacccggacgacccggttgacctgacctattcggaagactgcgagaagttcatcctcaaggtgccgacccggctgctggactcgatctgcgatgaacagcgctggcagcggcctgatgggggcgtgcggttcttgcgcaatcattaccggctggatgagctggatggcttcgtcaacctgctggccatggtttgtcatgaggcggaagtgagcgagtcgctgcccagggtgcaggggcactacagccaaattgtcgccagcaagttgctgaccctgatgaccaccaatatccgccgggagagcctgagtgcaccgcaggccggccttgagcgcattctcgattacatagagcgcaacctgaagttggagctgtcggccgaggtgctggcagagcaggcctgcatgagtttgcgttcgctgtatgcgctgtttgaacagcacctgagcaccacgcccaagcattacgtgcgccagcgcaagcttgagcgggtgcatgcgtgcctgagcgacccgacttgcggtgtgcgcagtgtgaccgaacttgccctggattacggcttctttcatttaggccggttttccgaggtgtatcggcagcaatttggcgagttgccttcgcagacgttcaagcgtcgggggtga

SEQ ID NO: 50 - DNA, 720 bp, synthetic construct
  source 1..720: mol_type = other DNA
atggtcattaaggcgcaaagcccggcgggtttcgcggaagagtacattattgaaagtatctggaataaccgcttccctcccgggactattttgcccgcagaacgtgaactttcagaattaattggcgtaacgcgtactacgttacgtgaagtgttacagcgtctggcacgagatggctggttgaccattcaacatggcaagccgacgaaggtgaataatttctgggaaacttccggtttaaatatccttgaaacactggcgcgactggatcacgaaagtgtgccgcagcttattgataatttgctgtcggtgcgtaccaatatttccactatttttattcgcaccgcgtttcgtcagcatcccgataaagcgcaggaagtgctggctaccgctaatgaagtggccgatcacgccgatgcctttgccgagctggattacaacatattccgcggcctggcgtttgcttccggcaacccgatttacggtctgattcttaacgggatgaaagggctgtatacgcgtattggtcgtcactatttcgccaatccggaagcgcgcagtctggcgctgggcttctaccacaaactgtcggcgttgtgcagtgaaggcgcgcacgatcaggtgtacgaaacagtgcgtcgctatgggcatgagagtggcgagatttggcaccggatgcagaaaaatctgccgggtgatttagccattcaggggcgataa

SEQ ID NO: 51 - DNA, 465 bp, synthetic construct
  source 1..465: mol_type = other DNA
atggaaaagaaattaccccgcattaaagcgctgctaacccccggcgaagtggcgaaacgcagcggtgtggcggtatcggcgctgcatttctatgaaagtaaagggttgattaccagtatccgtaacagcggcaatcagcggcgatataaacgtgatgtgttgcgatatgttgcaattatcaaaattgctcagcgtattggcattccgctggcgaccattggtgaagcgtttggcgtgttgcccgaagggcatacgttaagtgcgaaagagtggaaacagctttcgtcccaatggcgagaagagttggatcggcgcattcataccttagtggcgctgcgtgacgaactggacggatgtattggttgtggctgcctttcgcgcagtgattgcccgttgcgtaacccgggcgaccgcttaggagaagaaggtaccggcgcacgcttgctggaagatgaacaaaactaa

SEQ ID NO: 52 - DNA, 2679 bp, synthetic construct
  source 1..2679: mol_type = other DNA
atgctttcaccccatagcgccctcgcgtttcggggagacgaagcaggagatcggattgtcatgacccgccatagcttggaagactggtgtcggaggatgtctgaaaggatgtgggctcgtcagtgtttcattactcaagggtgtttagaccgctattgcctgagcgcttctgctacgcctgaatcaaaggtagttctgtatcgggccccgccaggctatggaaaatctgtacaagtggcctttgcagcaaacactcgcgaactggaacttgagagcgtcgtttacatcaatacccgatctttccctgactcatatggcgttaatgacagtttgtttgcagcgttagttttatatcaaatcgaaggagttgaatcttggcgcacgataaaaagtgacatcgacaccatcgaagctcttcgaaacgcactgtgtaatgcaaaaaagacgataagaatttgccttgatgggattggcgaggctaaacataccgcggatttgattgaggatttaatctgtgaaacaccgagcaatgtgaaatttttcattgcaccgagtcgcgtcggggcgttggcgcgtttgtctatgatgacgggtgttgtaacgtatggtgctgatgaacttgtatttacagaagaggaggttagtgaattatcgggtatgggtggccccgaagccagcaacatcataaaagccaccggtggatggccagcacttgtcaggcttatgtgccaaaccccaaacccaaatctccccgcagcgacttggccggaaactcgctcgtatttcagggacaacctcttaactgcattacccgatagtacgaggacctttctttgcaatgcagcgatgctcgaagtaatcagcgctgaatgctatggctatgtctataagacagaagaggccgaccgggaaattacgtttcttaacgaacattatgctcttttagctcccaccggaacttctggtgatcgtatggatatgcaccctgtgctgcgagagtatttgcgtagtttatttaacaccacccaacgagaacgtcgctcgtatgtactgaaacgggtcgcgttctggcattggcgtaggggagagtacctccattcaatcaatgcagctctggaagccagtgaccatcgttgggctcgcgtcgtaagtgacagcattattctggacgtggctctgaggcagggtgaaatcgaagtacttcgcacctggtttggaaaagtaccggctcgtacgataaaaaaaatcgctgccttgagcataggttacgcgtggattttgtatttcagccaacaagctcgtcaagcagaggaaatactggttagttcgcttcatccgagctccaggggggtgggcaatcttgatgaggaaggatggcgggaattggttaatgcgataggcaaagctacacaggatgaattactggagagtcaggttctctgtcaaaaatggatagactcctttggcgaccgtaacatggttggcaaaggcgcagcacttacttgccaggcattcattgcctccagtggacgccattttgaagagttggagcgattgtcccatcgagcggctgtggccatccagtcatccaatcaacattacgcttttatctggcttaaaactgcagaaattcaggctgaactgttcaagggtgacatcgcccgagcaatgagtgtgttgatccaggcgaataaaacagcagaaaagattcaggtgccaaaaacttttttaaataaaatgtttggcacccttgagttacaaatcctgcacgaacagaaccatcacctggttacccaggaatctgcacaggcttcgttcgatttcgctttgaactatggcgttacagatattctgtgggggtgtacacaggcctacagccgttttctgtaccaccaagggttccgagatagagccatggccttgttggaacaatcccgcttagcctcctgcgagcgagagctctctcgtctaaatatcctgactaaagttcagttagctgagtttactctcctgaacaacgaggaattgggtccacccatactccccgatgaatgtgagctgacgttcctgccaaatcaaaaccaggccattcaagcacgtatggccctggtaacctcaatgtatagactacggcttgggaaacagtttggtgtcgccgaaaaatatgccaaacaagcactacaaagcgccagcgcaatttcagacgctagaaccaggatcgccgcccactattgtcaggccctggcagcgtttgccctcggctcatcaaaatcggcgaagcgcatgattcttgatgcaaacctactttcggaacatttgaactgtcacttcacacggctctggatcaaagaagcgctactttctcttagcccccttgctcaggatctttttaacgatctgccagacgactctgaagctaaagctacgaaggatcccgatgtcagaatagaagactccagcgtgccacctgtgcccggcaatgcccactcaactatcacgataaagcagatttctctgttgaaatgcgtaagcaacggaatgaccaacagagagatcgctgaacggcttctcgtgacggaggataccgtcaaatggcatatgaagaagattttcagcgaacttaaagtaaccaaccgggttcaggcagtcactgaagctcgactgcgcggacttttatag

SEQ ID NO: 53 - DNA, 816 bp, synthetic construct
  source 1..816: mol_type = other DNA
atgccttctctcgatgcttttctagagcacccaaactcgcatgagaaaatacgtcaagatgactacattgcagggttagcaaaaggcttggctttactggaagcttttggaacagatcggcaacgactcaatgtcactcaagtggccgagcgaactggtattagtcgtacagcggcaaggcgttacctaaaaactttaaaatacttaggctatttagaaaccgatgaacattacttttggcttactcatcgcgtgctgcgcttttcgagttcttatctaagctctgcccacttgcctaaagttgctcaatcatttttaaatcttttgtgtgcacagacatctcttactttttcaattgtggtgttagacgataacgaagtggtgccgattgcccgtagttatttaccacagcaagataatttacgtgtcagcccttatggtatgcatttaggaaaccgactccctgcccatgccacttccacaggtaaagtattgctggcagcactacccgaagaagctcaacatacgtgggtaaaaacttacggtttaaagcgcttaacaccatttaccatagtagatgaatcaaagttttttgaagtgcttaagggtatttcgttatcggattattgtctttccaaagaagaacatgagcttggcgtaattgcgattgcagtgcccgtatttaacgcccaaggtcaagccatagcagcgcttaactgcatgtctcaaactaaccgtgtacaagaagattatttggttcagcaaattttgccattgctgcgcaacacggcaaatgaactgagaaatgtgatttaa

SEQ ID NO: 54 - DNA, 41 bp, synthetic construct
  source 1..41: mol_type = other DNA
ctttaagaaggagatatacatatgaacacgattaacatcgc

SEQ ID NO: 55 - DNA, 39 bp, synthetic construct
  source 1..39: mol_type = other DNA
tggtggtggtggtgctcgagttacgcgaacgcgaagtcc

SEQ ID NO: 56 - DNA, 44 bp, synthetic construct
  source 1..44: mol_type = other DNA
cttacattaattgcgttgcgcccgcttctagaggagctgttgac

SEQ ID NO: 57 - DNA, 45 bp, synthetic construct
  source 1..45: mol_type = other DNA
gatatttttgccgatccccattgatcttttctcctcttttcctcc

SEQ ID NO: 58 - DNA, 42 bp, synthetic construct
  source 1..42: mol_type = other DNA
taagaaggagatatacatcgaggatgaaacatggcatctatt

SEQ ID NO: 59 - DNA, 36 bp, synthetic construct
  source 1..36: mol_type = other DNA
cctgggcatgccgcttcagtggtggtggtggtggtg